(19) United States
(12) Patent Application Publication
(10) Pub. No.: US 2009/0197967 A1
(43) Pub. Date: Aug. 6, 2009

(54) COMPOSITIONS AND METHODS FOR TREATMENT OF OPHTHALMIC DISEASES AND DISORDERS

(76) Inventors: Ryo Kubota, Seattle, WA (US); Ahmad Fawzi, Bellevue, WA (US); Ian L. Scott, Monroe, WA (US); Vladimir A. Kuksa, Kenmore, WA (US)

Correspondence Address: WILSON SONSINI GOODRICH & ROSATI, 650 PAGE MILL ROAD, PALO ALTO, CA, 94304-1050, US

(21) Appl. No.: 12/162,476

(22) PCT Filed: Jan. 26, 2007
(86) PCT No.: PCT/US07/02330

§ 371 Date: Jan. 12, 2009

Related U.S. Application Data
(60) Provisional application No. 60/762,384, filed on Jan. 26, 2006.

(51) Int. Cl. A61K 31/135; A61K 31/13

(52) U.S. Cl. 514/646; 514/671; 514/659

(57) ABSTRACT

Provided herein are compositions and methods for treating ophthalmic diseases and disorders. Compositions comprising retinylamine derivative compounds provided herein are useful for treating and preventing ophthalmic diseases and disorders, including diabetic retinopathy diabetic maculopathy, diabetic macular edema, retinal ischemia, ischemia-reperfusion related retinal injury, and metabolic optic neuropathy.

### CROSS-REFERENCE TO RELATED APPLICATION

This application claims the benefit of U.S. Provisional Patent Application No. 60/762,384, filed Jan. 26, 2006, which is incorporated herein by reference in its entirety.

### BACKGROUND OF THE INVENTION

1. Field of the Invention

The present invention relates generally to compositions and methods for treating neurodegenerative diseases and disorders, particularly ophthalmic diseases and disorders. Provided herein are compositions comprising retinoid compounds, including retinylamine derivative compounds, that are useful for treating and preventing ophthalmic diseases and disorders, including diabetic retinopathy and macular degeneration.

2. Description of the Related Art

Neurodegenerative diseases, such as glaucoma, macular degeneration, diabetic retinopathy, and Alzheimer's disease, affect millions of patients throughout the world. Because the loss of quality of life associated with these diseases is considerable, drug research and development in this area is of great importance.

Macular degeneration affects between five and ten million patients in the United States, and it is the leading cause of blindness worldwide. Macular degeneration affects central vision and causes the loss of photoreceptor cells in the central part of retina called the macula. Macular degeneration can be classified into two types: dry type and wet type. The dry form is more common than the wet, with about 90% of age-related macular degeneration (ARMD) patients diagnosed with the dry form. The wet form of the disease and geographic atrophy, which is the end-stage phenotype of dry ARMD, lead to more serious vision loss. All patients who develop wet form ARMD previously had dry form ARMD for a prolonged period of time. The exact causes of age-related macular degeneration are still unknown. The dry form of ARMD may result from the aging and thinning of macular tissues and from deposition of pigment in the macula. In wet ARMD, new blood vessels grow beneath the retina and leak blood and fluid. This leakage causes the retinal cells to die, creating blind spots in central vision.

For the vast majority of patients who have the dry form of macular degeneration, no treatment is available. Because the dry form precedes development of the wet form of macular degeneration, intervention in disease progression of the dry form could benefit patients that presently have dry form and may delay or prevent development of the wet form.

Declining vision noticed by the patient or by an ophthalmologist during a routine eye exam may be the first indicator of macular degeneration. The formation of exudates, or “drusen,” underneath the Bruch's membrane of the macula is often the first physical sign that macular degeneration may develop. Symptoms include perceived distortion of straight lines and, in some cases, the center of vision appears more distorted than the rest of a scene; a dark, blurry area or “white-out” appears in the center of vision; and/or color perception changes or diminishes.

Different forms of macular degeneration may also occur in younger patients. Non-age related etiology may be linked to heredity, diabetes, nutritional deficits, head injury, infection, or other factors.

Glaucoma is a broad term used to describe a group of diseases that causes visual field loss, often without any other prevailing symptoms. The lack of symptoms often leads to a delayed diagnosis of glaucoma until the terminal stages of the disease. Prevalence of glaucoma is estimated to be three million in the United States, with about 120,000 cases of blindness attributable to the condition. The disease is also prevalent in Japan, which has four million reported cases. In other parts of the world, treatment is less accessible than in the United States and Japan, thus glaucoma ranks as a leading cause of blindness worldwide. Even if subjects afflicted with glaucoma do not become blind, their vision is often severely impaired.

The loss of peripheral vision is caused by the death of ganglion cells in the retina. Ganglion cells are a specific type of projection neuron that connects the eye to the brain. Glaucoma is often accompanied by an increase in intraocular pressure. Current treatment includes use of drugs that lower the intraocular pressure; however, lowering the intraocular pressure is often insufficient to completely stop disease progression. Ganglion cells are believed to be susceptible to pressure and may suffer permanent degeneration prior to the lowering of intraocular pressure. An increasing number of cases of normal tension glaucoma have been observed in which ganglion cells degenerate without an observed increase in the intraocular pressure. Because current glaucoma drugs only treat intraocular pressure, a need exists to identify new therapeutic agents that will prevent or reverse the degeneration of ganglion cells.

Recent reports suggest that glaucoma is a neurodegenerative disease, similar to Alzheimer's disease and Parkinson's disease in the brain, except that it specifically affects retinal neurons. The retinal neurons of the eye originate from diencephalon neurons of the brain. Though retinal neurons are often mistakenly thought not to be part of the brain, retinal cells are key components of the central nervous system, interpreting the signals from the light sensing cells.

Alzheimer's disease (AD) is the most common form of dementia among the elderly. Dementia is a brain disorder that seriously affects a person's ability to carry out daily activities. Alzheimer's is a disease that affects four million people in the United States alone. It is characterized by a loss of nerve cells in areas of the brain that are vital to memory and other mental functions. Some drugs can prevent AD symptoms for a finite period of time, but no drugs are available that treat the disease or completely stop the progressive decline in mental function. Recent research suggests that glial cells that support the neurons or nerve cells may have defects in AD sufferers, but the cause of AD remains unknown. Individuals with AD seem to have a higher incidence of glaucoma and macular degeneration, indicating that similar pathogenesis may underlie these neurodegenerative diseases of the eye and brain. (See Giasson et al., Free Radic. Biol. Med. 32:1264-75 (2002); Johnson et al., Proc. Natl. Acad. Sci. USA 99:11830-35 (2002); Dentchev et al., Mol. Vis. 9:184-90 (2003)).

Another leading cause of blindness is diabetic retinopathy, which is a complication of diabetes. Diabetic retinopathy occurs when diabetes damages blood vessels inside the retina. Non-proliferative retinopathy is a common, usually mild form that generally does not interfere with vision. Abnormalities are limited to the retina, and vision is impaired only if the macula is involved. If left untreated it can progress to proliferative retinopathy, the more serious form of diabetic retinopathy. Proliferative retinopathy occurs when new blood vessels proliferate in and around the retina. Consequently, bleeding into the vitreous, swelling of the retina, and/or retinal detachment may occur, leading to blindness.

Neuronal cell death underlies the pathology of these diseases. Unfortunately, very few compositions and methods that enhance retinal neuronal cell survival, particularly photoreceptor cell survival, have been discovered. A need therefore exists to identify and develop compositions that that can be used for treatment and prophylaxis of retinal diseases and disorders.

In vertebrate photoreceptor cells, a photon causes isomerization of the 11-cis-retinylidene chromophore to all-trans-retinylidene coupled to the visual opsin receptors. This photoisomerization triggers conformational changes of opsins, which, in turn, initiate the biochemical chain of reactions termed phototransduction (Filipek et al., Annu Rev Physiol 65: 851-79, 2003). Regeneration of the visual pigments requires that the chromophore be converted back to the 11-cis-configuration in the processes collectively called the retinoid (visual) cycle (reviewed in McBee et al., Prog Retin Eye Res 20:469-52, 2001). First, the chromophore is released from the opsin and reduced in the photoreceptor by retinol dehydrogenases. The product, all-trans-retinol, is trapped in the adjacent retinal pigment epithelium (RPE) in the form of insoluble fatty acid esters in subcellular structures known as retinosomes (Imanishi et al., J Cell Biol 164:373-8, 2004).

In Stargardt's disease (Allikmets et al., Nat Genet. 15:236-46, 1997), a disease associated with mutations in the ABCR transporter, the accumulation of all-trans-retinal may be responsible for the formation of a lipofuscin pigment, A2E, which is toxic towards retinal cells and causes retinal degeneration and, consequently, loss of vision (Mata and Travis, Proc Natl Acad Sci USA 97:7154-9, 2000; Weng et al, Cell 98:13-23, 1999). It was proposed that treating patients with an inhibitor of retinol dehydrogenases, 13-cis-RA (Accutane®, Roche), might prevent or slow down the formation of A2E and might have protective properties to maintain normal vision (Radu et al., Proc Natl Acad Sci USA 100:4742-7, 2003). 13-cis-RA (Isotretinoin, or Accutane®) inhibits 11-cis-RDH (Law and Rando, Biochem Biophys Res Commun 161:825-9, 1989) and is associated with induced night blindness, has been used to slow the synthesis of 11-cis-retinal through the inhibition of 11-cis-RDH. Others have proposed that 13-cis-RA works to prevent chromophore regeneration by binding RPE65, a protein essential for the isomerization process in the eye (Gollapalli and Rando, Proc. Natl. Acad. Sci. USA 101:10030-5, 2004). These investigators found that 13-cis-RA blocked the formation of A2E, and suggested that this treatment may inhibit lipofuscin accumulation and thus delay either the onset of visual loss in Stargardt's patients or the macular degeneration associated with lipofuscin accumulation. However, blocking the retinoid cycle and forming unliganded opsin (Van Hooser et al., J. Biol. Chem. 277:19173-82, 2002; Woodruff et al., Nat. Genet. 35:158-164, 2003) may result in more severe consequences and worsening of the patient's prognosis. Failure of the chromophore to form may lead to progressive retinal degeneration and in an extreme case will produce phenotype similar to Leber Congenital Amaurosis (LCA). This disease is a very rare childhood condition that affects children from birth or shortly thereafter. Furthermore treatment with 13-cis-RA is associated with induced night blindness.

A need exists in the art for an effective treatment for Stargardt's disease and age-related macular degeneration (AMD) without causing further unwanted side effects such as progressive retinal degeneration, LCA, or night blindness. A need also exists in the art for effective treatments for other ophthalmic diseases and disorders that adversely affect the retina.

### BRIEF SUMMARY OF THE INVENTION

Provided herein are retinylamine derivative compounds and compositions and methods for treating or preventing an ophthalmic disease or disorder, including a degenerative disease of the eye, which methods comprising administering to a subject an effective amount of a retinylamine derivative and a pharmaceutically acceptable carrier, vehicle, or excipient, which includes an opthalmologically acceptable carrier. Also provided herein are methods for preventing retinal cell (such as a retinal neuronal cell) degeneration (or enhance or prolong retinal cell survival or prolong retinal cell viability) in an eye or a subject. In other embodiments, methods are provided for restoring photoreceptor function in an eye of a subject, which methods comprise administering to the subject a retinylamine derivative as described in detail herein and a pharmaceutically acceptable carrier. These methods may slow chromophore flux in a retinoid cycle in the eye and restore photoreceptor function in the eye. In another embodiment, administration of the retinylamine derivative compound may inhibit an isomerization step of the retinoid cycle.

Provided herein is a method of treating or preventing an ophthalmic disease or disorder in a subject, wherein the ophthalmic disease or disorder is selected from diabetic retinopathy, diabetic maculopathy, diabetic macular edema, retinal ischemia, ischemia-reperfusion related retinal injury, and metabolic optic neuropathy, wherein the retinylamine derivative is a compound of formula I, provided herein. In certain embodiments, the method comprises a retinylamine derivative compound that has a substructure of formula I, (e.g., substructure of formula I(A) or I(B) and compounds (I(a)-I(j)). In certain embodiments, the retinylamine derivative is an all trans-isomer, a 9-cis-isomer, an 11-cis-isomer, a 13-cis-isomer, a 9,11-di-cis-isomer, a 9,13-di-cis-isomer, a 11,13-di-cis-isomer, or a 9,11,13-tri-cis-isomer. In a specific embodiment, the retinylamine derivative is 11-cis retinyl amine. In another specific embodiments, the retinylamine derivative is 9-cis retinylamine, 11-cis retinylamine, 13-cis retinylamine, or all trans retinylamine. In another particular embodiment, the retinylamine derivative has at least a 1+ charge at neutral pH (in presence of a counterion).

In other embodiments, a method is provided for treating or preventing an ophthalmic disease or disorder in a subject, wherein the ophthalmic disease or disorder is selected from diabetic retinopathy, diabetic maculopathy, diabetic macular edema, retinal ischemia, ischemia-reperfusion related retinal injury, and metabolic optic neuropathy, that comprises administering to the subject in need thereof a composition comprising a retinylamine derivative and a pharmaceutically acceptable carrier, wherein the retinylamine derivative is a compound of formula II, formula III, formula IV, or formula V, including a compound having a substructure of any one of the aforementioned formulas as described herein, including a retinylamine derivative compound of formula III that is a 11-cis locked retinylamine, and a compound having the structure of formula V(a)), all of which are described in detail herein. In particular embodiments, the retinylamine derivative has at least a 1+ charge at neutral pH (in presence of a counterion).

In certain embodiments of any of the aforementioned methods for treating an ophthalmic disease, accumulation of lipofuscin pigment is inhibited in an eye of the subject, and in specific embodiments, the lipofuscin pigment is N-retinylidene-N-retinyl-ethanolamine (A2E).

In other certain embodiments, the retinylamine derivative compounds having the structures I, II, III, IV, or V or any substructure described herein are used in methods for treating an ophthalmic disease that is selected from macular degeneration, glaucoma, retinal detachment, retinal blood vessel occlusion, hemorrhagic retinopathy, retinitis pigmentosa, retinopathy of prematurity, optic neuropathy, inflammatory retinal disease, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with Alzheimer's disease, a retinal disorder associated with multiple sclerosis, a retinal disorder associated with Parkinson's disease, a retinal disorder associated with viral infection, a retinal disorder related to light overexposure, and a retinal disorder associated with AIDS. In other specific embodiments, the methods of treating an ophthalmic disease or disorder excludes treating age related macular degeneration or Stargardt's disease (Stargardt's macular dystrophy).

In other specific embodiments, the retinylamine derivative compound is locally administered to an eye of the subject, which in certain embodiments is administered by eye drops, intraocular injection, or periocular injection. In another embodiment, the retinylamine derivative compound is orally administered in the subject. In another embodiment, a use of the retinylamine derivative compound having any one of structures 1, II, III, IV, or V or any substructure described herein is provided for the manufacture of a medicament for treating or preventing an ophthalmic disease or disorder. In certain specific embodiments, the use of the retinylamine derivative is for the manufacture of a medicament for treating diabetic retinopathy, retinal ischemia, diabetic macular edema, metabolic optic neuropathy, ischemia-reperfusion related retinal injury, or diabetic maculopathy.

In another embodiment, a method is provided for inhibiting degeneration of a retinal cell in an eye of subject in need thereof comprising administering to the subject a composition comprising a pharmaceutically acceptable carrier and a retinylamine derivative that is a compound having any one of structures I, II, III, IV, or V or any substructure thereof described herein as described herein. In certain embodiments, the method comprises a retinylamine derivative compound comprising compounds having substructures of formula I, (e.g., substructure of formula I(A) or I(B) and compounds (I(a)-I(j)). In certain embodiments, the retinylamine derivative is an all trans-isomer, a 9-cis-isomer, an 11-cis-isomer, a 13-cis-isomer, a 9,11-di-cis-isomer, a 9,13-di-cis-isomer, a 11,13-di-cis-isomer, or a 9,11,13-tri-cis-isomer. In a specific embodiment, the retinylamine derivative is 11-cis retinylamine. In another specific embodiments, the retinylamine derivative is 9-cis retinylamine, 11-cis retinylamine, 13-cis retinylamine, or all trans retinylamine. In another particular embodiment, the retinylamine derivative has at least a 1+ charge at neutral pH (in presence of a counterion).

In other embodiments, a method is provided for inhibiting degeneration of a retinal cell in an eye of a subject, comprising administering to the subject a composition that comprises a retinylamine derivative and a pharmaceutically acceptable carrier, wherein the retinylamine derivative is a compound of formula II, formula III, formula IV, or formula V, including a compound having a substructure of any one of the aforementioned formulas as described herein, and specific compounds (e.g., a retinylamine derivative compound of formula III that is 11-cis locked retinylamine; a compound having the structure of formula V(a)), all of which are described in detail herein. In particular embodiments, the retinylamine derivative has at least a 1+ charge at neutral pH (in presence of a counterion).

In certain embodiments of the aforementioned methods for inhibiting degeneration of a retinal cell in an eye of a subject, the retinal cell is a retinal neuronal cell or other mature retinal cell, such as a retinal pigmented epithelium (RPE) cell or a Müller glial cell. In a specific embodiment, the retinal neuronal cell is selected from an amacrine cell, ganglion cell, bipolar cell, horizontal cell, and a photoreceptor cell.

In other certain embodiments of the aforementioned methods for inhibiting degeneration of a retinal cell in an eye of a subject, the retinylamine derivative inhibits an isomerization step of the retinoid cycle. In another certain embodiment, the retinylamine derivative may slow chromophore flux in a retinoid cycle in the eye that may prevent degeneration of a retinal cell, wherein in certain embodiments, the retinal cell is a retinal neuronal cell. In other certain embodiments, the retinal neuronal cell is selected from a photoreceptor cell, amacrine cell, horizontal cell, bipolar cell, and a horizontal cell; in other certain embodiments the retinal neuronal cell is a photoreceptor cell.

In certain embodiments of any of the aforementioned methods for inhibiting degeneration of a retinal cell in an eye of a subject, the method further comprises inhibiting accumulation of lipofuscin pigment in an eye of the subject, and in specific embodiments, the lipofuscin pigment is N-retinylidene-N-retinyl-ethanolamine (A2E).

In another certain embodiment of any of the aforementioned methods for inhibiting degeneration of a retinal cell in an eye of a subject, inhibiting degeneration of a retinal cell in an eye of a subject by administering a composition comprising a pharmaceutical carrier and a retinylamine derivative as described herein is a treatment for an ophthalmic disease or disorder wherein the ophthalmic disease or disorder is selected from diabetic retinopathy, retinal ischemia, diabetic macular edema, metabolic optic neuropathy, ischemia-reperfusion related retinal injury, or diabetic maculopathy. In other embodiments, the ophthalmic disease or disorder is selected from macular degeneration, glaucoma, retinal detachment, retinal blood vessel occlusion, hemorrhagic retinopathy, retinitis pigmentosa, retinopathy of prematurity, optic neuropathy, inflammatory retinal disease, proliferative vitreoretinopathy, retinal dystrophy, hereditary optic neuropathy, metabolic optic neuropathy, Stargardt's macular dystrophy, Sorsby's fundus dystrophy, Best disease, uveitis, a retinal injury, a retinal disorder associated with Alzheimer's disease, a retinal disorder associated with multiple sclerosis, a retinal disorder associated with Parkinson's disease, a retinal disorder associated with viral infection, a retinal disorder related to light overexposure, and a retinal disorder associated with AIDS. In another certain embodiment, the ophthalmic disease is selected from glaucoma, diabetic retinopathy, diabetic maculopathy, retinal ischemia, diabetic macular edema, retinal detachment, retinal blood vessel occlusion, hemorrhagic retinopathy, retinitis pigmentosa, retinopathy of prematurity, optic neuropathy, inflammatory retinal disease, proliferative vitreoretinopathy, retinal dystrophy, ischemia-reperfusion related retinal injury, hereditary optic neuropathy, metabolic optic neuropathy, Sorsby's fundus dystrophy, Best disease, uveitis, a retinal injury, a retinal disorder associated with Alzheimer's disease, a retinal disorder associated with multiple sclerosis, a retinal disorder associated with Parkinson's disease, a retinal disorder associated with viral infection, a retinal disorder related to light overexposure, and a retinal disorder associated with AIDS. In a specific embodiment, the ophthalmic disease is diabetic retinopathy or diabetic maculopathy. In other specific embodiments, the methods of treating an ophthalmic disease or disorder excludes treating age related macular degeneration or Stargardt's disease. In other specific embodiments, the retinylamine derivative is locally administered to an eye of the subject, which in certain embodiments is administered by eye drops, intraocular injection, or periocular injection. In another embodiment, the retinylamine derivative is orally administered in the subject. In another embodiment, a use of the retinylamine derivative is provided for the manufacture of a medicament for treating or preventing an ophthalmic disease or disorder.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells and equivalents thereof known to those skilled in the art, and so forth. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

All U.S. patents, U.S. patent application publications, U.S. patent applications, foreign patents, foreign patent applications, and non-patent publications referred to in this specification and/or listed in the Application Data Sheet, are incorporated herein by reference, in their entireties.

### DETAILED DESCRIPTION OF THE INVENTION

The present invention relates to retinoid compounds, such as retinylamine derivatives, and compositions comprising such compounds that are useful for treating and preventing ophthalmic diseases and disorders, particularly including ophthalmic diseases and disorders that are associated with, or are sequelae of, metabolic diseases such as diabetes. Neurodegeneration of stressed retinal neuronal cells (e.g., amacrine, ganglion, bipolar cells, horizontal cells, and particularly photoreceptor cells) and other mature retinal cells, such as RPE and Müller glial cells, may be decreased or inhibited in these cells when the cells are exposed to the compounds described herein. Exposure of stressed retinal neuronal cells to the retinylamine derivative compounds described herein may result in prolonged survival, that is, survival of an increased number of retinal neuronal cells (for example, photoreceptor cells) than the number of cells that would survive in the absence of the compound. Methods are provided herein for using the retinylamine derivative compounds described herein to treat a subject who has or who is at risk of developing an ophthalmic disease or disorder, including but not limited to, diabetic retinopathy, diabetic maculopathy, diabetic macular edema, retinal ischemia, ischemia-reperfusion related retinal injury, and metabolic optic neuropathy.

These compounds may be used in methods for treating neurological diseases or disorders in general, and for treating degenerative diseases of the eye and brain in particular. The retinylamine compounds may be useful for treating, curing, preventing, ameliorating the symptoms of, or slowing, inhibiting, or stopping the progression of a neurodegenerative disease or disorder, particularly an ophthalmic disease or disorder. Representative ophthalmic diseases include but are not limited to macular degeneration (including dry form macular degeneration), glaucoma, diabetic retinopathy, diabetic maculopathy, diabetic macular edema, retinal detachment, retinal blood vessel (artery or vein) occlusion, hemorrhagic retinopathy, retinitis pigmentosa, retinopathy of prematurity, optic neuropathy, inflammatory retinal disease, proliferative vitreoretinopathy, retinal dystrophy, retinal ischemia, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with neurodegenerative diseases such as Alzheimer's disease, multiple sclerosis, and/or Parkinson's disease, a retinal disorder associated with viral infection, or a retinal disorder related to, or as a sequelae of, AIDS. A retinal disorder also includes retinal damage that is related to overexposure to light. In certain particular embodiments, use of the retinylamine compounds in the methods described herein for treating ophthalmic diseases or disorders excludes use of the compounds for treating age related macular degeneration and Stargardt's macular dystrophy.

Described herein are methods for treating or preventing an ophthalmic disease, such as a degenerative disease of the eye, comprising administering to a subject in need thereof a retinoid derivative, e.g., a retinylamine derivative, in a pharmaceutically acceptable carrier. Also provided herein are methods for preventing photoreceptor degeneration in a vertebrate eye or for restoring photoreceptor function comprising administering to a subject in need thereof a retinoid compound, e.g., a retinylamine derivative, in a pharmaceutically acceptable carrier, which without wishing to be bound by theory, may slow chromophore flux in a retinoid cycle in the eye.

After absorption of light and photoisomerization of 11-cis-retinal to all-trans retinal, regeneration of the visual chromophore is a critical step in restoring photoreceptors to their dark-adapted state. This regeneration process, called the retinoid (visual) cycle, takes place in the photoreceptor outer segments and retinal pigmented epithelium (RPE). Studies suggest that regeneration of the chromophore in the eye can occur through a retinyl carbocation intermediate and that positively charged retinoids can act as transition state analogs of the isomerization process (see, e.g., Golczak et al., Proc. Natl. Acad. Sci. USA 102:8162-67 (2005)). The isomerization process has not yet been fully characterized in molecular detail (see, e.g., Rando, Biochemistry 30:595-602 (1990); Kuksa et al., Vision Res 43:2959-81 (2003); Stecher et al., J Biol Chem 274:8577-85 (1999); McBee et al., Biochemistry 39:11370-80 (2000); Stecher and Palczewski, Methods Enzymol 316:330-44 (2000)).

Without wishing to be bound by any particular theory, molecular characterization of the isomerization process has been described by at least two hypotheses. The “isomerohydrolase” hypothesis proposes the existence of an enzyme that would utilize the energy of retinyl ester hydrolysis to carry out the endothermic isomerization reaction (Rando, Biochemistry 30:595-602, 1990). This mechanism entails a nucleophilic attack at the C₁₁ position of all-trans-retinyl palmitate with concurrent elimination of palmitate by alkyl cleavage. The complex rotates to reposition the C₁₁-C₁₂ bond into a new conformation followed by rehydration of the transition state of the chromophore-protein complex, leading to the production of 11-cis-retinol. Direct evidence is lacking for this mechanism, and its pros and cons have been extensively discussed (see, e.g., Kuksa et al., Vision Res. 43:2959-81, 2003). An alternative mechanism has been proposed, in which all-trans-retinyl esters are converted into an unidentified intermediate, which could be all-trans-retinol, a subpopulation of activated esters, or a retinoid intermediate not yet known in the art) (see, e.g., Stecher et al., J. Biol. Chem. 274:8577-85, 1999). This intermediate may then be converted to a retinyl carbocation, rehydrated in the transition state, and released as 11-cis-retinol (see, e.g., McBee et al., Biochemistry 39:11370-80, 2000)). Significant product formation in this endothermic reaction should only be seen in the presence of retinoid-binding proteins (see, e.g., Stecher and Palczewski, Methods Enzymol. 316:330-44, 2000), and studies indicate that the ratio of the isomers produced appears to be sensitive to the specificity of the retinoid-binding proteins (see, e.g., Stecher et al., J. Biol. Chem. 274:8577-85, 1999; McBee et al., Biochemistry 39:11370-80, 2000). In both mechanisms the pathway would progress via an alkyl cleavage, as observed experimentally (see, e.g., Kuksa et al., Vision Res. 43:2959-81, 2003).

While a retinylamine (Ret-NH₂) binds proteins in the RPE microsomes, it may not bind RPE65, a protein implicated in the isomerization reaction. Golczak et al. (supra) suggest that positively charged retinoid derivatives, e.g., retinylamine, can regulate chromophore flux more specifically than does 13-cis-retinoic acid (13-cis-RA). The compound 13-cis-RA has been proposed to treat symptoms of Stargardt's disease by slowing the retinoid cycle; however, the compound may adversely affect many other tissues than the eye. In addition, 13-cis-RA can spontaneously isomerize to the all-trans isomer, which in turn activates the nuclear receptors RXR and RAR. Ret-NH₂ does not interact at micromolar concentrations with RXR and RAR.

Without wishing to be bound by theory, 11-cis-retinylamine and other retinylamine compounds described herein, may inhibit, block, or in some manner interfere with the isomerization process, and are thus useful for treating ophthalmic diseases and disorders. 11-cis-retinylamine is prepared by reductive amination of 11-cis-retinal. The amine is a strong inhibitor of the isomerase, or isomerohydrolase, a protein involved in the visual cycle. In vivo inhibition of isomerase after light bleaching does not lead to the recovery of visual pigment chromophore, thus preventing the formation of retinals and increasing the amount of retinyl esters. The retinals are responsible for the accumulation of toxic lipofuscin pigment, A2E, which is believed to be highly toxic to retinal cells, contributing to retinal degeneration. Accordingly, and as described herein, retinylamine derivative compounds as described herein, such as 11-cis-retinylamine, may be used for treating any number of ophthalmic diseases and disorders as described herein.

“Retinoids” refers to a class of compounds consisting of four isoprenoid units joined in a head to tail manner. See IUPAC-IUB Joint Commission on Biochemical Nomenclature. All retinoids may be formally derived from a monocyclic parent compound containing five carbon-carbon double bonds and a functional group at the terminus of the acyclic portion. The basic retinoid structure is generally subdivided into three segments, namely (1) a polar terminal end (e.g., a terminal amine, alcohol, aldehyde or acid); (2) a conjugated side chain; and (3) a cyclohexenyl ring or a non-polar alkyl side chain. The basic structures of the most common natural retinoids are called retinol, retinaldehyde, and retinoic acid.

Retinylamine derivatives include positively charged retinoid derivatives, which refer to a retinoid class of compounds, with a positively charged substituent, for example, a positively charged nitrogen atom (such as present in a quaternary amine). The positively charged retinoid derivative may be positively charged via protonation or as a salt (for example, in the presence of a counterion, the compound may be positively charged at neutral pH). The retinylamine derivative compound may be positively charged when it is in a physiologically active state and/or when the compound is interacting with an enzyme at the enzymatic and/or substrate binding site. Positively charged substituents include onium compounds, which include (1) cations (with their counter-ions) that are derived by addition of a hydron (ion H+) to a mononuclear parent hydride of the nitrogen, chalcogen, and halogen families (e.g., ammonium (H₄N⁺); oxonium (H₃O⁺); fluoronium (H₃F⁺); phosphonium (H₄P⁺); sulfonium (H₃S⁺); chloronium (H₂Cl⁺); arsonium (H₄As⁺); selenonium (H₃Se⁺); bromonium (H₂Br⁺); stibonium (H₄Sb⁺); telluronium (H₃Te⁺); iodonium (H₂₁⁺); and bismuthonium (H₄Bi⁺)); (2) derivatives that are formed by substitution of the parent ion (see (1)) by univalent groups, wherein the number of substituted hydrogen atoms is indicated by the adjectives primary, secondary, tertiary, or quaternary; (3) derivatives that are formed by substitution of the parent ion (see (1)) by groups that have two or three free valencies on the same atom (e.g., R₂C═N⁺H₂ X⁻, which is an iminium compound) (see, e.g., IUPAC Compendium of Chemical Terminology, 2^{nd} ed. (1997)). Additional positively charged substituents include, but are not limited to, an amine, disubstituted imidazolium, trisubstituted imidazolium, pyridinium, pyrrolidinium, phosphonium, guanidinium, isouronium, iodonium, or sulfonium (for example SMe₃⁺I⁻) when these substituents are further protonated so that a positive charge is conferred (such as a protonated primary, secondary, or tertiary amine, or protonated disubstituted imidazolium etc.). Examples of positively charged retinoid derivative are retinylamine derivatives, including 11-cis-retinylamine, 13-cis-retinylamine, and 9-cis-retinylamine when the retinylamine derivatives are further protonated.

In certain embodiments, a “synthetic retinoid” comprises a retinoid compound, such as a retinylamine derivative, that is a “synthetic cis retinoid,” or a “synthetic cis retinylamine,” and in certain other embodiments, the synthetic retinoid comprises a retinoid compound that is a “synthetic trans retinoid” or a “synthetic trans retinylamine.” Synthetic retinoids include 11-cis-retinylamine derivatives, 13-cis-retinylamine derivatives, or 9-cis-retinylamine derivatives such as, for example, the following: acyclic retinylamines; retinylamines with modified polyene chain length, such as trienoic or tetraenoic retinylamines; retinylamines with substituted polyene chains, such as alkyl, halogen or heteroatom-substituted polyene chains; retinylamines with modified polyene chains, such as trans- or cis-locked polyene chains, or with, for example, allene or alkyne modifications; and retinylamines with ring modifications, such as heterocyclic, heteroaromatic or substituted cycloalkane or cycloalkene rings.

Methods are provided herein for treating or preventing an ophthalmic disease or disorder (including but not limited to diabetic retinopathy, diabetic maculopathy, diabetic macular edema, retinal ischemia, ischemia-reperfusion related retinal injury, and metabolic optic neuropathy) in a subject, which methods comprise administering to the subject in need thereof a retinylamine derivative having a structure of any one of formulas I-V and substructures thereof described in greater detail herein in a pharmaceutically acceptable carrier. Methods are also provided for inhibiting degeneration of a retinal cell (or enhancing or prolonging retinal cell survival or promoting retinal cell viability) in an eye of a subject comprising administering to the subject in need thereof a pharmaceutically acceptable carrier and a retinylamine derivative having a structure of any one of formulas I-V and substructures thereof as described herein.

In one embodiment of the method described herein for treating an ophthalmic disease or disorder in a subject in need thereof, the method comprises administering a composition comprising a pharmaceutically acceptable carrier and a retinylamine derivative that is a compound having the structure of formula I:

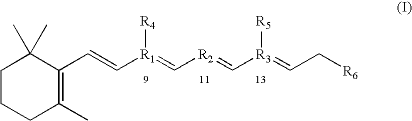

or a stereoisomer, prodrug, pharmaceutically acceptable salt, hydrate, solvate, acid salt hydrate, N-oxide or isomorphic crystalline form thereof,

wherein R₁ and R₃ are independently C or N⁺;

wherein R₂ is CH, N, or NR₇⁺;

wherein R₄ and R₅ are each the same or different, and independently H, saturated or unsaturated lower alkyl, C₃ to C₄ cycloalkyl, disubstituted imidazolium, trisubstituted imidazolium, pyridinium, pyrrolidinium, phosphonium, guanidinium, isouronium, iodonium, sulfonium, —CH₂—SR₇R₈⁺, —CH₂—NR₇R₈, —NR₇R₈, or —NR₇R₈R₉⁺;

wherein R₆ is H, saturated or unsaturated C₁ to C₁₄ alkyl, C₃ to C₁₀ cycloalkyl, halogen, heterocycle, phosphonium, guanidinium, isouronium, iodonium, sulfonium, CH₂—SR₇R₈⁺, —OR₇, —SR₇, —CH₂—NR₇R₈, —NR₇R₈, or —NR₇R₈R₉⁺;

wherein R₇, R₈, and R₉ are each the same or different and independently H, saturated or unsaturated lower alkyl, C₃ to C₄ cycloalkyl, —OH, or —OR₁₀, and wherein R₁₀ is a saturated lower alkyl;

with the proviso that the compound of formula I comprises at least one of the following:

(1) R₁ is N⁺;

(2) R₂ is N or NR₇⁺;

(3) R₃ is N⁺; and

(4) at least one of R₄, R₅, and R₆ is —NR₇R₈ or —NR₇R₈R₉⁺.

In certain embodiments, the retinylamine derivative compound has a structure of formula (I) wherein R₁ is N⁺; R₂ is N; or NR₇⁺; R₃ is N⁺. In other certain embodiments, at least one of R₄, R₅, and R₆ is —NR₇R₈ or —NR₇R₈R₉⁺. In another specific embodiment, R₆ is a heterocycle wherein the heterocycle is selected from disubstituted imidazolium, trisubstituted imidazolium, pyridinium, and pyrrolidinium.

In another specific embodiment, each of R₁ and R₃ is C, and R₂ is CH, and wherein at least one of R₄, R₅, and R₆ is —NR₇R₈ or —NR₇R₈R₉⁺. In still another specific embodiment, each of R₄ and R₅ is a lower alkyl and R₆ is —NR₇R₈ or —NR₇R₈R₉⁺. In a more specific embodiment, each of R₄ and R₅ is a methyl, or at least one of each of R₄ and R₅ is a methyl. In other specific embodiments, each of R₄ and R₅ is a lower alkyl and R₆ is —NR₇R₈ or —NR₇R₈R₉⁺.

In other specific embodiments, R₆ is a substituted C₁ to C₁₄ alkyl or substituted C₃ to C₁₀ cycloalkyl. In particular embodiments, the C₁ to C₁₄ alkyl or C₃ to C₁₀ cycloalkyl is substituted with NR₇R₈ or —NR₇R₈R₉⁺, and in other particular embodiments, wherein the substituent replaces a hydrogen atom at any one or more of the carbon atoms in the alkyl or cycloalkyl, including the carbon at the terminal end of an alkyl chain. In certain specific embodiments, R₇ is H and R₈ is hydrogen or a lower alkyl (i.e., C₁₋₆ alkyl, such as methyl (CH₃), ethyl, propyl, etc.).

In a specific embodiment, when the retinylamine derivative is positively charged, the compound of formula (I) is a salt and further comprises a counterion, X. In certain specific embodiments, X is an anion, for example, Cl, Br, I, SO₃H, or P(O)₂(OH)₂. In other specific embodiments, the retinylamine derivative has at least a I+charge at neutral pH, wherein in certain specific embodiments, at least one nitrogen atom carries a positive charge.

In certain embodiments, the retinylamine derivative has a substructure of formula I (referred to herein as substructure IA), wherein each of R₁ and R₃ is C and R₂ is CH; wherein R₄, R₅ and R₆ are defined above as for the structure of formula (I) (i.e., R₄ and R₅ are each the same or different and independently H, saturated or unsaturated lower alkyl, C₃ to C₄ cycloalkyl, disubstituted imidazolium, trisubstituted imidazolium, pyridinium, pyrrolidinium, phosphonium, guanidinium, isouronium, iodonium, sulfonium, —CH₂—SR₇R₈⁺, —CH₂—NR₇R₅, —NR₇R₉, or —NR₇R₈R₉⁺; and R₆ is H, saturated or unsaturated C₁ to C₁₄ alkyl, C₃ to C₁₀ cycloalkyl, halogen, heterocycle, phosphonium, guanidinium, isouronium, iodonium, sulfonium, CH₂—SR₇R₈⁺, —OR₇, —SR₇, —CH₂—NR₇R₈, —NR₇R₉, or —NR₇R₈R₉⁺) with the proviso that at least one of R₄, R₅, and R₆ is —NR₇R₈, or —NR₇R₈R₉⁺.

In certain embodiments, when the retinylamine derivative is positively charged, the certain substructure IA is a salt and further comprises a counterion, X. In certain specific embodiments, X is an anion, for example, Cl, Br, I, SO₃H, or P(O)₂(OH)₂. In other specific embodiments, the retinylamine derivative has at least a 1+ charge at neutral pH, wherein in certain specific embodiments, at least one nitrogen atom carries a positive charge.

In another certain embodiment, the retinylamine compound has the following substructure I(B), wherein each of R₁ and R₃ is C, and R₂ is CH and the retinylamine derivative compound has the following structure of formula I(B):

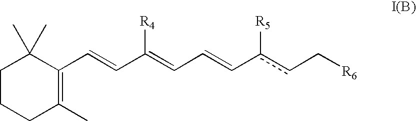

wherein R₄ and R₅ are each the same or different and independently H, saturated or unsaturated lower alkyl, C₃ to C₄ cycloalkyl, —CH₂—SR₇R₈⁺, —CH₂—NR₇R₈, —NR₇R₈, or —NR₇R₈R₉⁺;

wherein R₆ is H, saturated or unsaturated C₁ to C₁₄ alkyl, C₃ to C₁₀ cycloalkyl, halogen, heterocycle, —CH₂—SR₇R₈⁺, —OR₇, —SR₇, —CH₂—NR₇R₈, —NR₇R₈, or —NR₇R₈R₉⁺;

wherein R₇, R₈, and R₉ are each the same or different and independently H, saturated or unsaturated lower alkyl, C₃ to C₄ cycloalkyl, —OH, or —OR₁₀, wherein R₁₀ is a saturated lower alkyl;

with the proviso that at least one of R₄, R₅, and R₆ is —NR₇R₉, or —NR₇R₈R₉⁺.

In certain embodiments of the substructure of formula I(B), R₆ is a heterocycle selected from disubstituted imidazolium, trisubstituted imidazolium, pyridinium, and pyrrolidinium. In yet another specific embodiments, each of R₄ and R₅ is a lower alkyl and R₆ is —NR₇R₈ or —NR₇R₈R₉⁺. In a more specific embodiment, each of R₄ and R₅ is methyl, or at least one of R₄ and R₅ is methyl.

In a certain embodiment, in any one of the structures or substructures described above and herein, either one or both of R₄ and R₅ is a saturated or unsaturated lower alkyl (i.e., saturated C₁ to C₆ alkyl, C₂ to C₆ alkenyl, or C₂ to C₆ alkynyl). In other certain embodiments, R₆ is saturated C₁ to C₁₄ alkyl, C₁ to C₁₄ alkenyl, C₁ to C₁₄ alkylyl, or C₃ to C₁₄ branched alkyl. In another specific embodiment, any one or more of R₇, R₈, and R₉ is hydrogen or a saturated or unsaturated lower alkyl (i.e., saturated C₁ to C₆ alkyl, C₂ to C₆ alkenyl, or C₂ to C₆ alkynyl). In another specific embodiment, R₆ is —NH₂, or —NR₇R₈, wherein R₇ is H and R₈ is a lower alkyl (i.e., C₁₋₆ alkyl, such as methyl (CH₃), ethyl, propyl, etc.) or —OR₁₀, and wherein in another specific embodiment, R₁₀ is a lower alkyl (i.e., C₁₋₆ alkyl, such as methyl (CH₃), ethyl, propyl, etc.) and in specific embodiments, R₁₀ is CH₃. Further, as defined herein an alkyl, cycloalkyl, heterocycle group may be substituted or unsubstituted.

In certain embodiments, when the retinylamine derivative is positively charged, the certain substructure IB is a salt and further comprises a counterion, X. In certain specific embodiments, X is an anion, for example, Cl, Br, I, SO₃H, or P(O)₂(OH)₂. In other specific embodiments, the retinylamine derivative compound has at least a 1+ charge at neutral pH, wherein in certain specific embodiments, at least one nitrogen atom carries a positive charge.

In a specific embodiment, the retinylamine derivative is an all trans-isomer, a 9-cis-isomer; a 11-cis-isomer; a 13-cis-isomer; a 9,11-di-cis-isomer; a 9,13-di-cis-isomer; a 11,13-di-cis-isomer; or a 9,11,13-tri-cis-isomer. In certain embodiments, the retinylamine derivative has at least a 1+ charge at neutral pH, wherein in certain specific embodiments, at least one nitrogen atom carries a positive charge.

In certain specific embodiments, the retinoid compound has any one of the following structures I(a)-I(j).

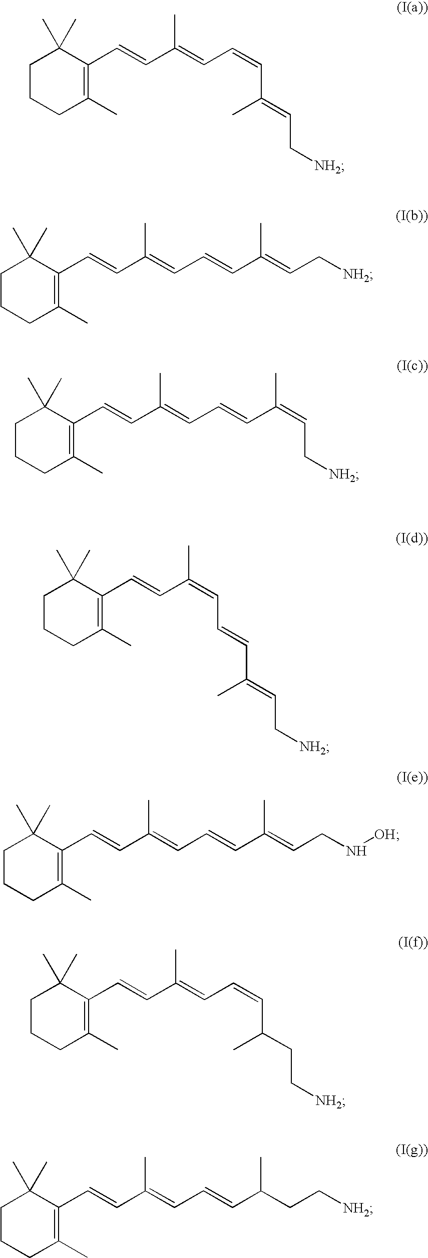

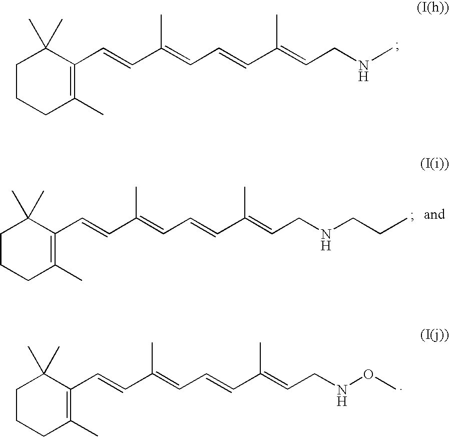

In a further embodiment, the retinylamine derivative is 11-cis retinylamine. In still other embodiments, the retinylamine derivative is selected from 9-cis retinylamine, 13-cis retinylamine, and all trans retinylamine.

In a certain embodiment, a retinylamine derivative compound described above and further herein may inhibit an isomerization step of the retinoid cycle.

In another embodiment of the method described herein for treating an ophthalmic disease or disorder (e.g. ophthalmic disease or disorder is selected from diabetic retinopathy, diabetic maculopathy, diabetic macular edema, retinal ischemia, ischemia-reperfusion related retinal injury, and metabolic optic neuropathy) in a subject in need thereof, the method comprises administering a pharmaceutically acceptable carrier and a retinylamine derivative, which is a compound having the structure of formula II:

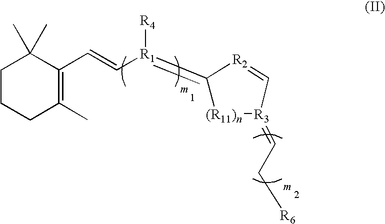

or a stereoisomer, prodrug, pharmaceutically acceptable salt, hydrate, solvate, acid salt hydrate, N-oxide or isomorphic crystalline form thereof,

wherein n is 1, 2, 3, or 4; and m₁ plus m₂ equals 1, 2, or 3; and

wherein R₁ and R₃ are each the same or different and independently C or N⁺; R₂ is CH, N, or NR₇⁺; and R₁₁ is C(H₂), N(R₇), or N(R₇R₈)⁺; R₄ is H, saturated or unsaturated lower alkyl, C₃ to C₄ cycloalkyl, disubstituted imidazolium, trisubstituted imidazolium, pyridinium, pyrrolidinium, phosphonium, guanidinium, isouronium, iodonium, sulfonium, —CH₂—SR₇R₈⁺, —CH₂—NR₇R₈, —NR₇R₈, or —NR₇R₈R₉⁺; R₆ is H, saturated or unsaturated C₁ to C₁₄ alkyl, C₃ to C₁₀ cycloalkyl, halogen, heterocycle, phosphonium, guanidinium, isouronium, iodonium, sulfonium, —CH₂—SR₇R₈⁺, —OR₇, —SR₇, —CH₂—NR₇R₈, —NR₇R₈, or NR₇R₈R₉⁺; R₇, R₈, and R₉ are each the same or different and independently H, saturated or unsaturated lower alkyl, C₃ to C₄ cycloalkyl, —OH, or —OR₁₀, and wherein R₁₀ is a saturated lower alkyl; with the proviso that the compound of formula II comprises at least one of the following:

(1) R₁ is N⁺;

(2) R₂ is N or NR₇⁺;

(3) R₃ is N⁺;

(4) R₁₁ is N(R₇), or N(R₇R₉)⁺; and

(5) at least one of R₄ and R₆ is —NR₇R₈ or —NR₇R₈R₉⁺.

In certain particular embodiments, the retinylamine derivative comprises a compound having a structure of formula (II) wherein R₁ is N⁺, and/or R₂ is N or N(R₇)⁺. In other specific embodiments, R₃ is N⁺; R₁₁ is N(R₇), or N(R₇R₈)⁺; and/or at least one of R₄ and R₆ is —NR₇R₈ or —NR₇R₈R₉⁺. In yet another specific embodiment, R₆ is a heterocycle selected from disubstituted imidazolium, trisubstituted imidazolium, pyridinium, and pyrrolidinium. In a particular embodiment, the method comprises administering a compound having a structure of formula (II) wherein each of R₁ and R₃ is C, R₂ is CH, and R₁₁ is C(H₂); and wherein at least one of R₄ and R₆ is —NR₇R₈ or —NR₇R₈R₉⁺.

In certain embodiments, when the retinylamine derivative compound is positively charged, the compound of formula (II) is a salt and further comprises a counterion, X. In certain specific embodiments, X is an anion, for example, Cl, Br, I, SO₃H, or P(O)₂(OH)₂. In other specific embodiments, the retinylamine derivative has at least a I+charge at neutral pH, wherein in certain specific embodiments, at least one nitrogen atom carries a positive charge.

In certain other embodiments, the retinylamine derivative has a substructure of formula II (referred to herein as substructure IIA) wherein R₁ and R₃ are C, R₂ is CH, and R₁₁ is C(H₂), and wherein R₄ and R₆ and all other substituents (i.e., R₇, R₈, and R₉ and R₁₀) are defined as above for the compound having the structure of formula (II), with the proviso that at least one of R₄ and R₆ is —NR₇R₈, or —NR₇R₈R₉⁺.

In another certain embodiment, the retinylamine derivative has a substructure of formula II (referred to herein as substructure IIB) wherein R₁ and R₃ are C, R₂ is CH, and R₁₁ is C(H₂); wherein R₄ is H, saturated or unsaturated lower alkyl, C₃ to C₄ cycloalkyl, —CH₂—SR₇R₈⁺, —CH₂—NR₇R₈, —NR₇R₈, or —NR₇R₈R₉⁺; wherein R₆ is H, saturated or unsaturated C₁ to C₁₄ alkyl, C₃ to C₁₀ cycloalkyl, halogen, heterocycle, —CH₂—SR₇R₈, —OR₇, —SR₇, —CH₂—NR₇R₈, —NR₇R₈, or —NR₇R₈R₉⁺; wherein R₇, R₈, and R₉ are each the same or different and independently H, saturated or unsaturated lower alkyl, C₃ to C₄ cycloalkyl, —OH, or —OR₁₀, and wherein R₁₀ is a saturated lower alkyl; and wherein at least one of R₄ and R₆ is —NR₇R₉ or —NR₇R₈R₉⁺.

In certain embodiments, R₄ is a saturated or unsaturated lower alkyl (i.e., saturated C₁ to C₆ alkyl, C₂ to C₆ alkenyl, or C₂ to C₆ alkynyl). In a more specific embodiment, R₄ is methyl. In other certain embodiments, R₆ is a saturated C₁ to C₁₄ alkyl, C₁ to C₁₄ alkenyl, C₁ to C₁₄ alkylyl, or C₃ to C₁₄ branched alkyl. In another certain embodiment, R₇, R₈, and R₉ are each the same or different and independently hydrogen or a saturated or unsaturated lower alkyl (i.e., saturated C₁ to C₆ alkyl, C₂ to C₆ alkenyl, or C₂ to C₆ alkynyl). Further, as defined herein an alkyl, cycloalkyl, heterocycle group may be substituted or unsubstituted. In another specific embodiment, R₆ is a heterocycle wherein the heterocycle is selected from disubstituted imidazolium, trisubstituted imidazolium, pyridinium, and pyrrolidinium.

In certain embodiments, when the retinylamine derivative is positively charged, any compound of substructure II(A) or II(B) is a salt and further comprises a counterion, X. In certain specific embodiments, X is an anion, for example, Cl, Br, I, SO₃H, or P(O)₂(OH)₂. In other specific embodiments, the retinylamine derivative has at least a I+charge at neutral pH, wherein in certain specific embodiments, at least one nitrogen atom carries a positive charge.

In another embodiment, a retinylamine derivative compound of formula II has the following substructure of formula III:

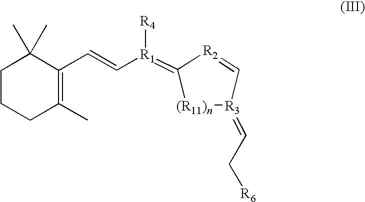

or a stereoisomer, prodrug, pharmaceutically acceptable salt, hydrate, solvate, acid salt hydrate, N-oxide or isomorphic crystalline form thereof,

wherein n is 1, 2, 3, or 4; and

wherein R₁ and R₃ are each the same or different and independently C or N⁺; R₂ is CH, N, or N(R₇)⁺; and R₁₁ is C(H₂), N(R₇), or N(R₇R₈)-4; R₄ is H, saturated or unsaturated lower alkyl, C₃ to C₄ cycloalkyl, disubstituted imidazolium, trisubstituted imidazolium, pyridinium, pyrrolidinium, phosphonium, guanidinium, isouronium, iodonium, sulfonium, —CH₂—SR₇R₈⁺, —CH₂—NR₇R₈, —NR₇R₈, or —NR₇R₈R₉⁺; R₆ is H, saturated or unsaturated C₁ to C₁₄ alkyl, C₃ to C₁₀ cycloalkyl, halogen, heterocycle, phosphonium, guanidinium, isouronium, iodonium, sulfonium, —CH₂—SR₇R₈⁺, —OR₇, —SR₇, —CH₂—NR₇R₈, —NR₇R₈, or —NR₇R₈R₉⁺; and wherein R₇, R₈, and R₉ are each independently H, saturated or unsaturated lower alkyl, C₃ to C₄ cycloalkyl, —OH, or —OR₁₀, and wherein R₁₀ is a saturated lower alkyl; with the proviso that the compound of formula III comprises at least one of the following:

(1) R₁ is N⁺;

(2) R₂ is N or N(R₇)⁺;

(3) R₃ is N⁺;

(4) R₁₁ is N(R₇), or N(R₇R₈)⁺; and

(5) at least one of R₄ and R₆ is —NR₇R₉ or —NR₇R₈R₉⁺.

In certain particular embodiments, the retinylamine derivative comprises a compound having a structure of formula (III) wherein R₁ is N⁺; R₂ is N or N(R₇⁺); R₃ is N⁺; R₁₁ is N(R₇), or N(R₇R₈)⁺; and/or at least one of R₄ and R₆ is —NR₇R₉ or —NR₇R₈R₉⁺.

In certain embodiments, the retinylamine derivative compound has a substructure of formula III (referred to herein as substructure III(A)), wherein each of R₁ and R₃ is C, R₂ is CH, and R₁₁ is C(H₂), and wherein R₄ and R₆ and all other substituents (i.e., R₇, R₈, R₉ and R₁₀) are defined as for the structure of formula (III), with the proviso that at least one of R₄ and R₆ is —NR₇R₈, or —NR₇R₈R₉⁺. In another specific embodiment, R₆ is a heterocycle selected from disubstituted imidazolium, trisubstituted imidazolium, pyridinium, and pyrrolidinium.

In another certain embodiment, the retinylamine derivative compound has a substructure of formula III (referred to herein as substructure III(B)), wherein each of R₁ and R₃ is C, R₂ is CH, and R₁₁ is C(H₂); wherein R₄ is H, lower alkyl, C₃ to C₄ cycloalkyl, —CH₂—SR₇R₈⁺, —CH₂—NR₇R₈, —NH₂, or —NR₇R₈R₉⁺; wherein R₆ is H, saturated or unsaturated C₁ to C₁₄ alkyl, C₃ to C₁₀ cycloalkyl, halogen, heterocycle, —CH₂—SR₇R₈⁺, —OR₇, —SR₇, —CH₂—NR₇R₈, —NR₇R₈, or —NR₇R₈R₉⁺; wherein R₇, R₈, and R₉ are independently, H, saturated or unsaturated lower alkyl, C₃ to C₄ cycloalkyl, —OH, or —OR₁₀, and wherein R₁₀ is a saturated lower alkyl; with the proviso that at least one of R₄ and R₆ is —NR₇R₉, or —NR₇R₈R₉⁺. In another specific embodiment, each of R₁ and R₃ is C, R₂ is CH, and R₁₁ is C(H₂), and at least one of R₄ and R₆ is —NR₇R₈ or —NR₇R₈R₉⁺.

In a certain embodiments, in any of the structures or substructures of formula III, formula IIIA, or formula IIIB, R₄ is hydrogen or a saturated or unsaturated lower alkyl (i.e., saturated C₁ to C₆ alkyl, C₂ to C₆ alkenyl, or C₂ to C₆ alkynyl). In a more specific embodiment, R₄ is a methyl. In other certain embodiments, R₆ is saturated C₁ to C₁₄ alkyl, C₁ to C₁₄ alkenyl, C₁ to C₁₄ alkylyl, or C₃ to C₁₄ branched alkyl. In another certain embodiment, R₇, R₈, and R₉ are each the same or different and independently hydrogen or a saturated or unsaturated lower alkyl (i.e., saturated C₁ to C₆ alkyl, C₂ to C₆ alkenyl, or C₂ to C₆ alkynyl). Further, as defined herein the alkyl, cycloalkyl, heterocycle groups may be substituted or unsubstituted. In another specific embodiment, R₆ is a heterocycle wherein the heterocycle is selected from disubstituted imidazolium, trisubstituted imidazolium, pyridinium, and pyrrolidinium.

In a specific embodiment, the positively charged retinoid derivative is 11-cis locked retinylamine (i.e., rotation is restricted at the double bond to the 11-cis geometric isomer, such as by incorporation into a ring).

In certain embodiments, when the retinylamine derivative is positively charged, any compound of structure III, substructure III(A), or II(B) is a salt and further comprises a counterion, X. In certain specific embodiments, X is an anion, for example, Cl, Br, I, SO₃H, or P(O)₂(OH)₂. In other specific embodiments, the retinylamine derivative has at least a 1+ charge at neutral pH, wherein in certain specific embodiments, at least one nitrogen atom carries a positive charge.

In yet another embodiment of the method described herein for treating an ophthalmic disease or disorder (e.g., diabetic retinopathy, diabetic maculopathy, diabetic macular edema, retinal ischemia, ischemia-reperfusion related retinal injury, and metabolic optic neuropathy) in a subject in need thereof, comprises administering to the subject a composition comprising a retinylamine derivative and a pharmaceutically acceptable carrier, wherein the retinylamine derivative is a compound of formula IV:

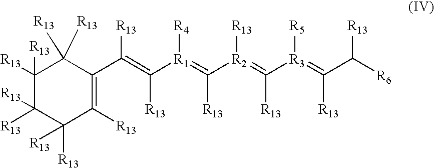

or a stereoisomer, prodrug, pharmaceutically acceptable salt, hydrate, solvate, acid salt hydrate, N-oxide or isomorphic crystalline form thereof,

wherein each R₁₃ is independently hydrogen, saturated or unsaturated C₁ to C₁₄ alkyl, C₃ to C₁₀ cycloalkyl, halogen, heterocycle, —OR₁₄, —SR₁₄, or —NR₁₄R₁₅, and wherein R₁₄ and R₁₅ are each independently H or saturated lower alkyl;

R₁, R₂, and R₃ are each independently C or N⁺;

R₄ and R₅ are each independently H, saturated or unsaturated lower alkyl, C₃ to C₄ cycloalkyl, disubstituted imidazolium, trisubstituted imidazolium, pyridinium, pyrrolidinium, phosphonium, guanidinium, isouronium, iodonium, sulfonium, —CH₂—SR₇R₈⁺, —CH₂—NR₇R₈, —NR₇R₈, or —NR₇R₈R₉⁺;

R₆ is H, saturated or unsaturated C₁ to C₁₄ alkyl, C₃ to C₁₀ cycloalkyl, halogen, heterocycle, phosphonium, guanidinium, isouronium, iodonium, sulfonium, —CH₂—SR₇R₈⁺, —OR₇, —SR₇, —CH₂—NR₇R₈, —NR₇R₈, or —NR₇R₈R₉⁺;

R₇, R₈, and R₉ are each the same or different and independently H, saturated or unsaturated lower alkyl, C₃ to C₄ cycloalkyl, —OH, or —OR₁₀, and wherein R₁₀ is saturated lower alkyl;

and with the proviso that the compound of formula IV comprises at least one of the following:

(1) at least one of R₁, R₂, and R₃ is N⁺; and

(2) at least one of R₄, R₅, and R₆ is —NR₇R₉ or —NR₇R₈R₉⁺.

In certain particular embodiments, the retinylamine derivative comprises a compound having a structure of formula (IV) wherein at least one of R₁, R₂, and R₃ is N⁺; and/or at least one of R₄, R₅, and R₆ is —NR₇R₈ or —NR₇R₈R₉⁺. In another particular embodiment, R₆ is a heterocycle selected from disubstituted imidazolium, trisubstituted imidazolium, pyridinium, pyrrolidinium.

In certain embodiments, the retinylamine derivative has a substructure of formula IV referred to herein as formula IV(A), wherein each of R₁, R₂, and R₃ is C; and wherein R₁₃, R₄, R₅, and R₆ and other substituents (i.e., R₇, R₅, R₉, R₁₀, R₁₄ and R₁₅) are defined as above for the structure of formula IV; with the proviso that at least one of R₄, R₅, and R₆ is —NR₇R₈, or —NR₇R₈R₉⁺.

In another certain embodiment, the retinylamine derivative has a substructure of formula IV referred to herein as formula IV(B), wherein each R₁₃ is independently hydrogen, saturated or unsaturated C₁ to C₁₄ alkyl, C₃ to C₁₀ cycloalkyl, halogen, heterocycle, —OR₁₄, —SR₁₄, or —NR₁₄R₁₅, and wherein R₁₄ and R₁₅ are each independently H or saturated lower alkyl; wherein R₁, R₂, and R₃ are each C; wherein R₄ and R₅ are each independently H, C₁ to C₆ alkyl, C₃ to C₄ cycloalkyl, —CH₂—SR₇R₈⁺, —CH₂—NR₇R₈, —NR₇R₈, or —NR₇R₈R₉⁺; wherein R₆ is H, saturated or unsaturated C₁ to C₁₄ alkyl, C₃ to C₁₀ cycloalkyl, halogen, heterocycle, —CH₂—SR₇R₈⁺, —OR₇, —SR₇, —CH₂—NR₇R₈, —NR₇R₈, or —NR₇R₈R₉⁺; wherein R₇, R₅, and R₉ are each independently H, saturated or unsaturated lower alkyl, C₃ to C₄ cycloalkyl, —OH, or —OR₁₀, and wherein R₁₀ is saturated lower alkyl; with the proviso that at least one of R₄, R₅, and R₆ is —NR₇R₈ or —NR₇R₈R₉⁺. Further, as defined herein the alkyl, cycloalkyl, heterocycle groups may be substituted or unsubstituted.

In other certain embodiments, in any of the structures or substructures of formula IV, formula IV(A), or formula IV(B), R₄ and R₅ are each the same or different and independently hydrogen or a substituted or unsubstituted, saturated or unsaturated lower alkyl (i.e., saturated C₁ to C₆ alkyl, C₂ to C₆ alkenyl, or C₂ to C₆ alkynyl). In a more specific embodiment, each of R₄ and R₅ is a methyl, or at least one of each of R₄ and R₅ is a methyl. In other certain embodiments, each R₁₃ is the same or different and independently hydrogen or a substituted or unsubstituted, saturated C₁ to C₁₄ alkyl, C₁ to C₁₄ alkenyl, C₁ to C₁₄ alkylyl, or C₃ to C₁₄ branched alkyl. In yet another certain embodiment, each R₁₃ is the same or different and independently a substituted or unsubstituted, saturated or unsaturated lower alkyl (i.e., saturated C₁ to C₆ alkyl, C₂ to C₆ alkenyl, or C₂ to C₆ alkynyl). In still other certain embodiments, R₆ is substituted or unsubstituted saturated C₁ to C₁₄ alkyl, C₁ to C₁₄ alkenyl, C₁ to C₁₄ alkylyl, or C₃ to C₁₄ branched alkyl. In still another embodiment, R₆ is a heterocycle wherein the heterocycle is selected from disubstituted imidazolium, trisubstituted imidazolium, pyridinium, and pyrrolidinium. In another certain embodiment, R₇, R₈, and/or R₉ is hydrogen or a substituted or unsubstituted, saturated or unsaturated lower alkyl (i.e., saturated C₁ to C₆ alkyl, C₂ to C₆ alkenyl, or C₂ to C₆ alkynyl). In another particular embodiment, at least one of R₁, R₂, and R₃ and at least one of the carbon atoms to which each is attached is absent such that the polyene chain has three, four, five, six, or seven carbon atoms.

In a specific embodiment, the retinylamine derivative is an all trans-isomer, a 9-cis-isomer, an 1′-cis-isomer, a 13-cis-isomer, a 9,11-di-cis-isomer, a 9,13-di-cis-isomer, and an 11,13-di-cis-isomer, or a 9,11,13-tri-cis-isomer. In certain embodiments, when the retinylamine derivative is positively charged, wherein the retinylamine derivative is any compound of structure IV, including substructures described herein such as a substructure of formula IV(A) and a substructure of formula IV(B), the retinylamine derivative is a salt and further comprises a counterion, X. In certain specific embodiments, X is an anion, for example, Cl, Br, I, SO₃H, or P(O)₂(OH)₂. In other specific embodiments, the retinylamine derivative has at least a 1+ charge at neutral pH, wherein in certain specific embodiments, at least one nitrogen atom carries a positive charge.

In another embodiment, the method described herein for treating an ophthalmic disease or disorder (e.g., diabetic retinopathy, diabetic maculopathy, diabetic macular edema, retinal ischemia, ischemia-reperfusion related retinal injury, or metabolic optic neuropathy), in a subject comprises administering to the subject a composition comprising a retinylamine derivative and a pharmaceutically acceptable carrier, wherein the retinylamine derivative is a compound of formula V:

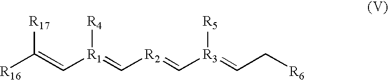

or a stereoisomer, prodrug, pharmaceutically acceptable salt, hydrate, solvate, acid salt hydrate, N-oxide or isomorphic crystalline form thereof,
wherein each of R₁₆ and R₁₇ is the same or different and independently substituted or unsubstituted lower alkyl, hydroxyl, alkoxy, —NR₇R₈, —NR₇R₈R₉⁺, or —NHC(═O)R₇; R₁ and R₃ are each independently C or N⁺; R₂ is CH, N, or NR₇⁺; R₄ and R₅ are each the same or different and independently H, saturated or unsaturated lower alkyl, C₃ to C₄ cycloalkyl, disubstituted imidazolium, trisubstituted imidazolium, pyridinium, pyrrolidinium, phosphonium, guanidinium, isouronium, iodonium, sulfonium, —CH₂—SR₇R₈⁺, —CH₂—NR₇R₈, —NR₇R₈, or —NR₇R₈R₉⁺; R₆ is H, C₁ to C₁₄ alkyl, C₃ to C₁₀ cycloalkyl, halogen, heterocycle, phosphonium, guanidinium, isouronium, iodonium, sulfonium, —CH₂—SR₇R₈⁺, —OR₇, —SR₇, —CH₂—NR₇R₈, —NR₇R₈, or —NR₇R₈R₉⁺; R₇, R₈, and R₉ are independently H, saturated or unsaturated lower alkyl, C₃ to C₄ cycloalkyl, —OH, or —OR₁₀, and wherein R₁₀ is a saturated lower alkyl; with the proviso that the compound of formula V comprises at least one of the following:

(1) R₁ is N⁺;

(2) R₂ is N or NR₉⁺;

(3) R₃ is N⁺; and

(4) at least one of R₄, R₅, and R₆ is —NR₇R₈ or —NR₇R₈R₉⁺.

In certain particular embodiments, the retinylamine derivative comprises a compound having a structure of formula (V) wherein R₁ is N⁺; R₂ is N or NR₇⁺; and/or R₃ is N⁺; and/or at least one of R₄, R₅, and R₆ is —NR₇R₈, or —NR₇R₈R₉⁺. In a particular embodiment, each of R₁ and R₃ is C and R₂ is CH; and at least one of R₄, R₅, and R₆ is —NR₇R₈ or —NR₇R₈R₉⁺. In other certain embodiments, R₆ is a heterocycle selected from disubstituted imidazolium, trisubstituted imidazolium, pyridinium, pyrrolidinium.

In certain embodiments, the retinylamine derivative compound has a substructure of formula V referred to herein as formula V(A), wherein R₁ and R₃ are C and R₂ is CH; and wherein R₁₆, R₁₇, R₄, R₅, R₆, R₇, R₈, and R₉ are defined as above for the structure of formula (V); with the proviso that at least one of R₄, R₅, and R₆ is —NR₇R₈ or —NR₇R₈R₉⁺.

In another certain embodiment, the retinylamine derivative compound has a substructure of formula V referred to herein as formula V(B), wherein each of R₁₆ and R₁₇ is independently substituted or unsubstituted lower alkyl, hydroxyl, alkoxy, —NR₇R₈, —NR₇R₈R₉⁺, or —NHC(═O)R₇; each of R₁ and R₃ is C and R₂ is CH; R₄ and R₅ are each the same or different and independently H, saturated or unsaturated lower alkyl, C₃ to C₄ cycloalkyl, —CH₂—SR₇R₈⁺, —CH₂—NR₇R₈, —NR₇R₈, or —NR₇R₈R₉⁺; R₆ is H, saturated or unsaturated C₁ to C₁₄ alkyl, C₃ to C₁₀ cycloalkyl, halogen, heterocycle, —CH₂—SR₇R₈⁺, —OR₇, —SR₇, —CH₂—NR₇R₈, —NR₇R₈, or —NR₇R₈R₉⁺; R₇, R₈, and R₉ are each the same or different and independently H, saturated or unsaturated lower alkyl, C₃ to C₄ cycloalkyl, —OH, or —OR₁₀, wherein R₁₀ is saturated lower alkyl; and with the proviso that at least one of R₄, R₅, and R₆ is —NR₇R₉ or —NR₇R₈R₉⁺.

In a certain embodiments, in any of the structures or substructures of formula V, formula V(A), or formula V(B), each of R₁₆ and R₁₇ is the same or different and independently hydrogen or a substituted or unsubstituted lower alkyl, wherein the lower alkyl is saturated or unsaturated (i.e., substituted or unsubstituted saturated C₁ to C₆ alkyl, substituted or unsubstituted C₂ to C₆ alkenyl, or substituted or unsubstituted C₂ to C₆ alkynyl). In a further embodiment, the substituted or unsubstituted lower alkyl is a substituted or unsubstituted branched lower alkyl. In yet another certain embodiment, each of R₄ and R₅ is the same or different and independently hydrogen or a saturated or unsaturated lower alkyl (i.e., saturated C₁ to C₆ alkyl, C₂ to C₆ alkenyl, or C₂ to C₆ alkynyl). In still other certain embodiments, R₆ is substituted or unsubstituted, saturated C₁ to C₁₄ alkyl, C₁ to C₁₄ alkenyl, C₁ to C₁₄ alkylyl, or C₃ to C₁₄ branched alkyl. In still another embodiment, R₆ is a heterocycle wherein the heterocycle is selected from disubstituted imidazolium, trisubstituted imidazolium, pyridinium, and pyrrolidinium. In another certain embodiment, each of R₇, R₈ and R₉ is the same or different and independently hydrogen or a saturated or unsaturated lower alkyl (i.e., saturated C₁ to C₆ alkyl, C₂ to C₆ alkenyl, or C₂ to C₆ alkynyl).

In a specific embodiment the retinylamine derivative compound is 10-ethyl-3,7-dimethyl-dodeca-2,4,6,8-tetraenylamine, which has the following structural formula (V(a)):

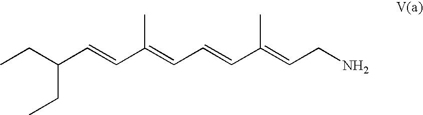

In certain embodiments, when the retinylamine derivative is positively charged, wherein the retinylamine derivative is any compound of structure V, including substructures described herein such as a substructure of formula V(A) and a substructure of formula V(B), the retinylamine derivative is a salt and further comprises a counterion, X. In certain specific embodiments, X is an anion, for example, Cl, Br, I, SO₃H, or P(O)₂(OH)₂. In other specific embodiments, the retinylamine derivative has at least a I+charge at neutral pH, wherein in certain specific embodiments, at least one nitrogen atom carries a positive charge.

In certain embodiments of the aforementioned methods for treating an ophthalmic disease by administering any one of the retinylamine derivative compounds described herein comprises inhibiting (i.e., preventing, decreasing, slowing, retarding in a statistically or biologically significant manner) degeneration of a retinal cell in an eye of a subject. A retinal cell includes a retinal neuronal cell or other mature retinal cell, such as a retinal pigmented epithelium (RPE) cell or a Müller glial cell. In a specific embodiment, the retinal neuronal cell is an amacrine cell, ganglion cell, bipolar cell, horizontal cell, or a photoreceptor cell. In a more specific embodiment, the methods described herein inhibit (i.e., prevent, decrease, slow, retard in a statistically or biologically significant manner) degeneration of a photoreceptor cell.

In other certain embodiments of the aforementioned methods for treating or preventing an ophthalmic disease or disorder and for inhibiting degeneration of a retinal cell in an eye of a subject, the retinylamine derivative may inhibit or block an isomerization step of the retinoid cycle. In another certain embodiment, the retinylamine derivative may slow (reduce, inhibit, retard) chromophore flux in a retinoid cycle in the eye, thereby preventing degeneration of a retinal cell. In certain embodiments, the retinal cell is a retinal neuronal cell. In other certain embodiments, the retinal neuronal cell is selected from a photoreceptor cell, amacrine cell, horizontal cell, bipolar cell, and a horizontal cell; in other certain particular embodiments the retinal neuronal cell is a photoreceptor cell.

In certain embodiments of any of the aforementioned methods for treating an ophthalmic disease or disorder and/or inhibiting degeneration of a retinal cell in an eye of a subject using any one or more of the retinylamine derivatives described herein, the retinylamine derivative may inhibit (i.e., prevent, reduce, decrease) accumulation of lipofuscin pigment in an eye of the subject. In a specific embodiment, the lipofuscin pigment is N-retinylidene-N-retinyl-ethanolamine (A2E).

### Chemistry Definitions

As used herein, the term disubstituted imidazolium means a positively charged imidazolyl ring that bears two non-H substituents, for example at least one hydrogen atom on each of two carbon atoms is replaced, at least one hydrogen atom on each of one carbon atom and one nitrogen atom is substituted, or at least one hydrogen atom on each of the two nitrogen atoms is replaced. As used herein the term trisubstituted imidazolium refers to a positively charged imidazole ring that bears three non-H substituents, for example, at least one hydrogen atom on each of the three carbon atoms is replaced, at least one hydrogen atom on one carbon atom and the two nitrogen atom is substituted, or at least one hydrogen atom on two carbon atoms and one nitrogen atom is substituted.

As used herein, alkyl, aryl, arylalkyl, homocycle, cycloalkyl, heterocycle, and heterocyclealkyl includes a substituted or unsubstituted alkyl, aryl, arylalkyl, homocycle, cycloalkyl, heterocycle, and heterocyclealkyl, respectively. The term “substituted” in the context of a substituted alkyl, aryl, arylalkyl, heterocycle, and heterocyclealkyl means that at least one hydrogen atom of the alkyl, aryl, arylalkyl, homocycle, cycloalkyl, heterocycle, and heterocyclealkyl moiety is replaced with a substituent. In the case of an oxo substituent (“═O”) two hydrogen atoms are replaced. The at least one hydrogen atom that is replaced includes a hydrogen atom of any one of the carbon atoms of an alkyl or cycloalkyl, or heterocyclealkyl.

A “substituent” as used herein includes oxo, halogen, hydroxy, cyano, nitro, amino, alkylamino, dialkylamino, alkyl, alkoxy, thioalkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NR₂C(═O)OR_{b} —NRₐSO₂R_{b}, —ORₐ, C(═O)Rₐ —C(═O)ORₐ, —C(═O)NRₐR_{b}, —OC(═O)NRₐR_{b}, —SH, —SRₐ, —SORₐ, —S(═O)₂Rₐ, —OS(═O)₂Rₐ —S(═O)₂NRₐR_{b} and —S(═O)₂ORₐ, wherein Rₐ and R_{b} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

Representative substituents include (but are not limited to) alkoxy (i.e., alkyl-O—, e.g., methoxy, ethoxy, propoxy, butoxy, pentoxy), aryloxy (e.g., phenoxy, chlorophenoxy, tolyloxy, methoxyphenoxy, benzyloxy, alkyloxycarbonylphenoxy, alkyloxycarbonyloxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkyloxycarbonyl-phenylthio), amino (e.g., amino, mono- and di-C₁-C₃ alkanylamino, methylphenylamino, methylbenzylamino, C₁-C₃ alkanylamido, acylamino, carbamamido, ureido, guanidino, nitro and cyano). Moreover, any substituent may have from 1-5 further substituents attached thereto.

“Alkyl” means a straight chain or branched, noncyclic or cyclic, unsaturated or saturated aliphatic hydrocarbon containing from 1 to 20 carbon atoms, and in certain embodiments from 1 to 14 carbon atoms. A lower alkyl has the same meaning as alkyl but contains from 1 to 6 carbon atoms. Representative saturated straight chain alkyls include methyl, ethyl, n-propyl, n-butyl, n-pentyl, n-hexyl, and the like. Saturated branched alkyls include isopropyl, sec-butyl, isobutyl, tert-butyl, isopentyl, and the like. Representative saturated cycloalkyls (cyclic alkyls) include cyclopropyl, cyclobutyl, cyclopentyl, cyclohexyl, —CH₂cyclopropyl, —CH₂cyclobutyl, —CH₂cyclopentyl, —CH₂cyclohexyl, and the like, while unsaturated cyclic alkyls include cyclopentenyl and cyclohexenyl, and the like. Cycloalkyls, also referred to as “homocyclic rings,” include di- and poly-homocyclic rings such as decalin and adamantyl. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. Representative straight chain and branched alkynyls include acetylenyl, propynyl, 1-butynyl, 2-butynyl, 1-pentynyl, 2-pentynyl, 3-methyl-1 butynyl, and the like.

“Heteroalkyl,” which includes heteroalkanyl, heteroalkenyl, heteroalkanyl, refers to an alkyl group, as defined herein, in which one or more of the carbon atoms (and any associated hydrogen atoms) are each independently replaced with the same or different heteroatoms or heteroatomic groups. Typical heteroatoms or heteroatomic groups that can be included in these groups include —O—, —S—, —O—O—, —S—S—, —O—S—, —O—S—O—, —O—NR′—, —NR′—, —NR′—S—S, —NR′—NR′—, —N═N—, —N═N—NR′—, —P(═O)₂—, —O—P(═O)₂—, —S(═O)₂—, and the like, and combinations thereof, including —NR′—S(═O)₂—, where each R′ is independently selected from hydrogen, alkyl, alkanyl, alkenyl, alkynyl, aryl, arylalkyl, heteroaryl and heteroarylalkyl, as defined herein. One example of a heteroatom is —NR′— wherein R′ is hydrogen (amino); another heteroatomic group is a disulfide —S—S—.

“Aryl” means an aromatic carbocyclic moiety such as phenyl or naphthyl (1- or 2-naphthyl).

“Arylalkyl” means an alkyl having at least one alkyl hydrogen atom replaced with an aryl moiety, such as —CH₂-phenyl, —CH═CH-phenyl, —C(CH₃)═CH-phenyl, and the like.

“Heteroaryl” means an aromatic heterocycle ring of 5 to 10 members and having at least one heteroatom selected from nitrogen, oxygen, and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring, which is either saturated, unsaturated, or aromatic, and which contains from 1 to 4 heteroatoms independently selected from nitrogen, oxygen, and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

“Homocycle” (also referred to herein as “homocyclic ring”) means a saturated or unsaturated (but not aromatic) carbocyclic ring containing from 3-7 carbon atoms, such as cyclopropane, cyclobutane, cyclopentane, cyclohexane, cycloheptane, cyclohexene, and the like.

“Halogen” means fluoro, chloro, bromo, and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Pharmaceutically acceptable salt” includes both acid and base addition salts. A pharmaceutically acceptable salt of structures I-V as well as of substructures thereof is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Methods of making synthetic retinoid compounds and derivatives are disclosed in, for example, the following references: Anal. Biochem. 272:232-42, 1999; Angew, Chem. 36:2089-93, 1997; Biochemistry 14:3933-41, 1975; Biochemistry 21:384-93, 1982; Biochemistry 28:2732-39, 1989; Biochemistry 33:408-16, 1994; Biochemistry 35:6257-62, 1996; Bioorganic Chemistry 27:372-82, 1999; Biophys. Chem. 56:31-39, 1995; Biophys. J. 56:1259-65, 1989; Biophys. J. 83:3460-6, 2002; Chemistry 7:4198-204, 2001; Chemistry (Europe) 5:1172-75, 1999; FEBS 158:1, 1983; J. American Chem. Soc. 104:3214-16, 1982; J. Am. Chem. Soc. 108:6077-78, 1986; J. Am. Chem. Soc. 109:6163, 1987; J. Am. Chem. Soc. 112:7779-82, 1990; J. Am. Chem. Soc. 119:5758-59, 1997; J. Am. Chem. Soc. 121:5803-04, 1999; J. American Chem. Soc. 123:10024-29, 2001; J. American Chem. Soc. 124:7294-302, 2002; J. Biol. Chem. 276:26148-53, 2001; J. Biol. Chem. 277:42315-24, 2004; J. Chem. Soc.—Perkin T. 1:1773-77, 1997; J. Chem. Soc.—Perkin T 1:2430-39, 2001; J. Org. Chem. 49:649-52, 1984; J. Org. Chem. 58:3533-37, 1993; J. Physical Chemistry B 102:2787-806, 1998; Lipids 8:558-65; Photochem. Photobiol. 13:259-83, 1986; Photochem. Photobiol. 44:803-07, 1986; Photochem. Photobiol. 54:969-76, 1991; Photochem. Photobiol. 60:64-68 (1994); Photochem. Photobiol. 65:1047-55, 1991; Photochem. Photobiol. 70:111-15, 2002; Photochem. Photobiol. 76:606-615, 2002; Proc. Natl. Acad. Sci. USA 88:9412-16, 1991; Proc. Natl. Acad. Sci. USA 90:4072-76, 1993; Proc. Natl. Acad. Sci. USA 94:13442-47, 1997; and Proc. R. Soc. Lond. Series B, Biol. Sci. 233(1270):55-76, 1988 (the disclosures of which are incorporated by reference herein).

Retinyl esters can be formed by methods known in the art such as, for example, by acid-catalyzed esterification of a retinol with a carboxylic acid, by reaction of retinal with carboxylic acid in the presence of coupling reagents such as dicyclohexylcarbodiimide, as similar, or by Mitsunobu reaction between retinol and carboxylic acid in the presence of triphenylphosphine and diethyl(isopropyl)azodicarboxylate, by reaction of an acyl halide with a retinol, by base-catalyzed reaction of acid anhydride with retinol, by transesterification of a retinyl ester with a carboxylic acid, by reaction of a primary halide with a carboxylate salt of a retinoic acid, or the like. In an exemplary embodiment, retinyl esters can be formed by acid-catalyzed esterification of a retinol with a carboxylic acid, such as, acetic acid, propionic acid, butyric acid, valeric acid, caproic acid, caprylic acid, pelargonic acid, capric acid, lauric acid, oleic acid, stearatic acid, palmitic acid, myristic acid, linoleic acid, succinic acid, fumaric acid or the like. In another exemplary embodiment, retinyl esters can be formed by reaction of an acyl halide with a retinol (see, e.g., Van Hooser et al., Proc. Natl. Acad. Sci. USA, 97:8623-28, 2000). Suitable acyl halides include, for example, acetyl chloride, palmitoyl chloride, or the like.

Retinyl ethers can be formed by methods known in the art, such as for example, reaction of a retinol with a primary alkyl halide.

In certain embodiments, trans-retinoids can be isomerized to cis-retinoids by exposure to UV light. For example, all-trans-retinal, all-trans-retinol, all-trans-retinyl ester or all-trans-retinoic acid can be isomerized to 9-cis-retinal, 9-cis-retinol, 9-cis-retinyl ester or 9-cis-retinoic acid, respectively. trans-Retinoids can be isomerized to 9-cis-retinoids by, for example, exposure to a UV light having a wavelength of about 365 nm, and substantially free of shorter wavelengths that cause degradation of cis-retinoids, as further described herein.

Retinyl acetals and hemiacetals can be prepared, for example, by treatment of 9-cis- and 11-cis-retonals with alcohols in the presence of acid catalysts. Water formed during reaction is removed, for example by Al₂O₃ of a molecular sieve. Retinyl oximes can be prepared, for example, by reaction of a retinal with hydroxylamine, O-methyl- or O-ethylhydroxyl amine, or the like.

The compounds employed in the methods described herein may exist in prodrug form. “Prodrug” is intended to include any covalently bonded carrier that releases the active parent drug, for example, wherein the active parent drug is a compound as described herein including retinylamine derivative compounds having a structure as set forth in any one of Formula I, II, III, IV, or V, or any substructure described herein when such prodrug is administered to a subject. Since prodrugs are known to enhance numerous desirable qualities of pharmaceuticals (e.g., solubility, bioavailability, manufacturing, etc.) the compounds used in the methods may, if desired, be delivered in prodrug form. Thus, the methods described herein include delivery of a retinylamine compound as a prodrug. Prodrugs of the compounds described herein may be prepared by modifying functional groups present in the compound in such a way that the modifications are cleaved, either in routine manipulation or in vivo within the subject being treated, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, or phenethyl esters.

Examples of prodrugs of retinylamines further include, but are not limited to, an amide derivative, thioamide derivative, carbamate derivative, thiocarbamate derivative, imide derivative, sulphonamide derivative, imine derivative, protonated imine derivative, isocyanate derivative, or isothiocyanate derivative of retinylamine. The prodrug can be, for example, a retinylamide, a retinylthioamide, a retinylcarbamate, or a retinylthiocarbamate.

In general, the compounds used in the reactions described herein may be made according to organic synthesis techniques known to those skilled in this art, starting from commercially available chemicals and/or from compounds described in the chemical literature. “Commercially available chemicals” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hanover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

Methods known to one of ordinary skill in the art may be identified through various reference books and databases. Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the retinylamine derivative compounds described herein is P. H. Stahl & C. G. Wermuth “Handbook of Pharmaceutical Salts”, Verlag Helvetica Chimica Acta, Zurich, 2002.

### Treatment of Ophthalmic Diseases and Disorders

The methods described herein using the above-described retinylamine derivative compounds and compositions comprising the compounds may be used for treating ophthalmic diseases and disorders that are associated with, or are sequelae of, metabolic diseases such as diabetes. The retinylamine derivative compounds described herein may therefore be useful for treating a subject who has or who is at risk of developing an ophthalmic disease or disorder including but not limited to diabetic retinopathy, diabetic maculopathy, diabetic macular edema, retinal ischemia, ischemia-reperfusion related retinal injury ischemia-reperfusion injury (such as that caused by transplant, surgical trauma, hypotension, thrombosis or trauma injury), and metabolic optic neuropathy.

These methods are useful for treating a subject who has an ophthalmic disease or disorder such as macular degeneration, glaucoma, retinal detachment, retinal blood vessel occlusion, hemorrhagic or hypertensive retinopathy, retinitis pigmentosa, retinopathy of prematurity, optic neuropathy, inflammatory retinal disease, proliferative vitreoretinopathy, retinal dystrophy, traumatic injury to the optic nerve (such as by physical injury, excessive light exposure, or laser light), hereditary optic neuropathy, neuropathy due to a toxic agent or caused by adverse drug reactions or vitamin deficiency, Stargardt's macular dystrophy, Sorsby's fundus dystrophy, Best disease, uveitis, a retinal disorder associated with Alzheimer's disease, a retinal disorder associated with multiple sclerosis; a retinal disorder associated with viral infection (wherein the virus is cytomegalovirus or herpes simplex virus), a retinal disorder associated with Parkinson's disease, a retinal disorder associated with AIDS, or other forms of progressive retinal atrophy or degeneration. In a specific embodiment, the disease or disorder is diabetic retinopathy, diabetic macular edema, retinal ischemia, or diabetic maculopathy. In another specific embodiment, the disease or disorder results from mechanical injury, chemical or drug-induced injury, thermal injury, radiation injury, light injury, laser injury. These methods are also useful for preventing ophthalmic injury from environmental factors such as light-induced oxidative retinal damage, laser-induced retinal damage, etc.

As described herein, a subject may be treated for ophthalmic diseases or disorders that are associated with or are sequelae of a metabolic disease such as diabetes, which includes diabetic retinopathy, diabetic macular edema, and diabetic maculopathy. Diabetes increases the permeability of blood vessel walls beneath the retina, allowing fluids and fatty exudates to accumulate in the macula. This accumulation causes macular edema, destabilizes RPE membranes, and causes abnormal blood vessel function, leading to light-exacerbated vision loss. Preventing the accumulation of these exudates (or phototoxic constituents, such as A2E) may protect the diabetic retina from degeneration.

In one embodiment, the method inhibits (i.e., prevents, reduces, slows, abrogates, minimizes) accumulation of a lipofuscin pigment in the eye. In another embodiment, a method is provided for inhibiting (i.e., preventing, reducing, slowing, abrogating, minimizing) N-retinylidene-N-retinylethanolamine (A2E) accumulation in the eye. The ophthalmic disease may result, at least in part, from lipofuscin pigment accumulation and/or from accumulation of N-retinylidene-N-retinylethanolamine (A2E) in the eye. Accordingly, in certain embodiments, methods are provided for inhibiting or preventing accumulation of lipofuscin pigment and/or A2E in the eye of a subject. These methods comprise administering to the subject a composition comprising a pharmaceutically acceptable carrier and a retinylamine derivative compound as described in detail herein, including a compound having the structure as set forth in any one of formulas I-V, substructures thereof, and retinylamine compounds described herein.

By way of background, accumulation of the pigment lipofuscin in retinal pigment epithelium (RPE) cells has been linked to progression of retinal diseases that result in blindness, including age-related macular degeneration (De Laey et al., Retina 15:399-406 (1995)). Lipofuscin granules are autofluorescent lysosomal residual bodies (also called age pigments). The major fluorescent species of lipofuscin is A2E (an orange-emitting fluorophore), which is a positively charged Schiff-base condensation-product formed by all-trans retinaldehyde with phosphatidylethanolamine (2:1 ratio) (see, e.g., Eldred et al., Nature 361:724-6 (1993); see also, Sparrow, Proc. Natl. Acad. Sci. USA 100:4353-54 (2003)). Much of the indigestible lipofuscin pigment is believed to originate in photoreceptor cells; deposition in the RPE occurs because the RPE internalize membranous debris that is discarded daily by the photoreceptor cells. Formation of this compound is not believed to occur by catalysis by any enzyme, but rather A2E forms by a spontaneous cyclization reaction. In addition, A2E has a pyridinium bisretinoid structure that once formed cannot be enzymatically degraded. Lipofuscin, and thus A2E, accumulate with aging of the human eye and also accumulate in a juvenile form of macular degeneration called Stargardt's disease.

A2E may induce damage to the retina via several different mechanisms. At low concentrations, A2E inhibits normal proteolysis in lysosomes (Holz et al., Invest. Opthalmol. Vis. Sci. 40:737-43 (1999)). At higher, sufficient concentrations, A2E may act as a positively charged lysosomotropic detergent, dissolving cellular membranes, and may alter lysosomal function, release proapoptotic proteins from mitochondria, and ultimately kill the RPE cell (see, e.g., Eldred et al., supra; Sparrow et al., Invest. Opthalmol. Vis. Sci. 40:2988-95 (1999); Holz et al., supra; Finneman et al., Proc. Natl. Acad. Sci. USA 99:3842-347 (2002); Suter et al., J. Biol. Chem. 275:39625-30 (2000)). A2E is phototoxic and initiates blue light-induced apoptosis in RPE cells (see, e.g., Sparrow et al., Invest. Opthalmol. Vis. Sci. 43:1222-27 (2002)). Upon exposure to blue light, photooxidative products of A2E are formed (e.g., epoxides) that damage cellular macromolecules, including DNA (Sparrow et al., J. Biol. Chem. 278(20):18207-13 (2003)). A2E self-generates singlet oxygen that reacts with A2E to generate epoxides at carbon-carbon double bonds (Sparrow et al., supra). Generation of oxygen reactive species upon photoexcitation of A2E causes oxidative damage to the cell, often resulting in cell death. An indirect method of blocking formation of A2E by inhibiting biosynthesis of the direct precursor of A2E, all-trans-retinal, has been described (see U.S. Patent Application Publication No. 2003/0032078). However, the usefulness of the method described therein is limited because generation of all-trans retinal is an important component of the visual cycle. Other therapies described include neutralizing damage caused by oxidative radical species by using superoxide-dismutase mimetics (see, e.g., U.S. Patent Application Publication No. 2004/0116403) and inhibiting A2E-induced cytochrome C oxidase in retinal cells with negatively charged phospholipids (see, e.g., U.S. Patent Application Publication No. 2003/0050283).

The retinylamine derivative compounds described herein may be useful for inhibiting, (i.e., preventing, reducing, slowing, retarding, or decreasing) accumulation (i.e., deposition) of A2E in the RPE. Without wishing to be bound by theory, because the RPE is critical for the maintenance of the integrity of photoreceptor cells, preventing, reducing, or inhibiting damage to the RPE may inhibit degeneration (enhance the survival or increase cell viability) of retinal neuronal cells, particularly, photoreceptor cells. Compounds that bind specifically to or interact with A2E or that affect A2E formation or accumulation may also reduce, inhibit, prevent, or decrease one or more toxic effects of A2E that result in retinal neuronal cell (including a photoreceptor cell) damage, loss, or neurodegeneration, or in some manner cause a decrease retinal neuronal cell viability. Such toxic effects include induction of apoptosis, self-generation of singlet oxygen and generation of oxygen reactive species; self-generation of singlet oxygen to form A2E-epoxides that induce DNA lesions, thus damaging cellular DNA and inducing cellular damage; dissolving cellular membranes; altering lysosomal function; and effecting release of proapoptotic proteins from mitochondria.

A subject in need of such treatment may be a human or may be a non-human primate or other animal (i.e., veterinary use) who has developed symptoms of an ophthalmic disease or disorder or who is at risk for developing an ophthalmic disease or disorder. Examples of non-human primates and other animals include but are not limited to farm animals, pets, and zoo animals (e.g., horses, cows, buffalo, llamas, goats, rabbits, cats, dogs, chimpanzees, orangutans, gorillas, monkeys, elephants, bears, large cats, etc.).

Also provided herein are methods for inhibiting (i.e., reducing, slowing, retarding, preventing) degeneration of retinal neuronal cells and enhancing or prolonging retinal neuronal cell survival (or prolonging cell viability) comprising administering to a subject in need thereof a composition comprising a pharmaceutically acceptable carrier and at least one of the retinylamine derivative compounds described in detail herein, including a compound having any one of the structures set forth in formulas I-V, substructures thereof, and specific retinylamine compounds described herein. A retinal neuronal cell includes a photoreceptor cell, a bipolar cell, a horizontal cell, a ganglion cell, and an amacrine cell. In another embodiment, methods are provided for enhancing or prolonging survival or inhibiting degeneration of a mature retinal cell such as a RPE cell or a Müller glial cell. In another embodiment, a method for preventing or inhibiting photoreceptor degeneration in an eye of a subject or a method for restoring photoreceptor function in an eye of a subject is provided that comprises administering to the subject in need thereof a composition comprising a retinylamine compound as described herein and a pharmaceutically or acceptable carrier. Such methods comprise administering to a subject in need thereof, a pharmaceutically acceptable carrier and a retinylamine derivative described herein, including a compound having any one of the structures set forth in formulas I-V or substructures thereof described herein. In certain embodiments, the retinylamine derivative is a positively charged retinoid compound as described herein. Without wishing to be bound by theory, the retinylamine derivative may inhibit an isomerization step of the retinoid cycle and/or may slow chromophore flux in a retinoid cycle in the eye.

The ophthalmic disease may result, at least in part, from lipofuscin pigment accumulation and/or from accumulation of N-retinylidene-N-retinylethanolamine (A2E) in the eye. Accordingly, in certain embodiments, methods are provided for inhibiting or preventing accumulation of lipofuscin pigment and/or A2E in the eye of a subject. These methods comprise administering to the subject a composition comprising a pharmaceutically acceptable carrier and a retinylamine derivative compound as described in detail herein, including a compound having the structure as set forth in any one of formulas I-V or substructures thereof.

A retinylamine compound can be administered to a subject who has an excess of a retinoid in an eye (e.g., an excess of 11-cis-retinol or 11-cis-retinal), an excess of retinoid waste products or intermediates in the recycling of all-trans-retinal, or the like. The eye typically comprises a wild-type opsin protein. Methods of determining endogenous retinoid levels in a vertebrate eye, and an excess or deficiency of such retinoids, are disclosed in, for example, U.S. Patent Application Publication No: 2005/0159662 (the disclosure of which is incorporated by reference herein in its entirety). Other methods of determining endogenous retinoid levels in a subject, which is useful for determining whether levels of such retinoids are above the normal range, and include for example, analysis by high pressure liquid chromatography (HPLC) of retinoids in a biological sample from a subject. For example, retinoid levels can be determined in a biological sample that is a blood sample (which includes serum or plasma) from a subject. A biological sample may also include vitreous fluid, aqueous humor, intraocular fluid, or tears.

For example, a blood sample can be obtained from a subject and different retinoid compounds and levels of one or more of the retinoid compounds in the sample can be separated and analyzed by normal phase high pressure liquid chromatography (HPLC) (e.g. with a HP 1100 HPLC and a Beckman, Ultrasphere-Si, 4.6 mm×250 mm column using 10% ethyl acetate/90% hexane at a flow rate of 1.4 ml/minute). The retinoids can be detected by, for example, detection at 325 nm using a diode-array detector and HP Chemstation A.03.03 software. An excess in retinoids can be determined, for example, by comparison of the profile of retinoids (i.e., qualitative, e.g., identity of specific compounds, and quantitative, e.g., the level of each specific compound) in the sample with a sample from a normal subject. Persons skilled in the art who are familiar with such assays and techniques and will readily understand that appropriate-controls are included.

As used herein, increased or excessive levels of endogenous retinoid, such as 11-cis-retinol or 11-cis-retinal, refer to levels of endogenous retinoid higher than those found in a healthy eye of a vertebrate of the same species. Administration of a synthetic retinylamine derivative can reduce or eliminate the requirement for endogenous retinoid.

### Retinal Cells

The retina of the eye is a thin, delicate layer of nervous tissue. The major landmarks of the retina are the area centralis in the posterior portion of the eye and the peripheral retina in the anterior portion of the eye. The retina is thickest near the posterior sections and becomes thinner near the periphery. The area centralis is located in the posterior retina and contains the fovea and foveola and, in primates, contains the macula. The foveola contains the area of maximal cone density and, thus, imparts the highest visual acuity in the retina. The foveola is contained within the fovea, which is contained within the macula.

The peripheral or anterior portion of the retina increases the field of vision. The peripheral retina extends anterior to the equator of the eye and is divided into four regions: the near periphery (most posterior), the mid-periphery, the far periphery, and the ora serrata (most anterior). The ora serrata denotes the termination of the retina.

The term neuron (or nerve cell) as understood in the art and used herein denotes a cell that arises from neuroepithelial cell precursors. Mature neurons (i.e., fully differentiated cells from an adult) display several specific antigenic markers. Neurons may be classified functionally into three groups: (1) afferent neurons (or sensory neurons) that transmit information into the brain for conscious perception and motor coordination; (2) motor neurons that transmit commands to muscles and glands; and (3) interneurons that are responsible for local circuitry; and (4) projection interneurons that relay information from one region of the brain to another region and therefore have long axons. Interneurons process information within specific subregions of the brain and have relatively shorter axons. A neuron typically has four defined regions: the cell body (or soma); an axon; dendrites; and presynaptic terminals. The dendrites serve as the primary input of information from other cells. The axon carries the electrical signals that are initiated in the cell body to other neurons or to effector organs. At the presynaptic terminals, the neuron transmits information to another cell (the postsynaptic cell), which may be another neuron, a muscle cell, or a secretory cell. The retina is composed of several types of neuronal cells. As described herein, the types of retinal neuronal cells that may be cultured in vitro by this method include photoreceptor cells, ganglion cells, and interneurons such as bipolar cells, horizontal cells, and amacrine cells. Photoreceptors are specialized light-reactive neural cells and comprise two major classes, rods and cones. Rods are involved in scotopic or dim light vision, whereas photopic or bright light vision originates in the cones by the presence of trichromatic pigments. Many neurodegenerative diseases that result in blindness, such as macular degeneration, retinal detachment, retinitis pigmentosa, diabetic retinopathy, etc, affect photoreceptors.

Extending from their cell bodies, the photoreceptors have two morphologically distinct regions, the inner and outer segments. The outer segment lies furthermost from the photoreceptor cell body and contains disks that convert incoming light energy into electrical impulses (phototransduction). The outer segment is attached to the inner segment with a very small and fragile cilium. The size and shape of the outer segments vary between rods and cones and are dependent upon position within the retina. See Eye and Orbit, 8^{th} Ed., Bron et al., (Chapman and Hall, 1997).

Ganglion cells are output neurons that convey information from the retinal interneurons (including horizontal cells, bipolar cells, amacrine cells) to the brain. Bipolar cells are named according to their morphology, and receive input from the photoreceptors, connect with amacrine cells, and send output radially to the ganglion cells. Amacrine cells have processes parallel to the plane of the retina and have typically inhibitory output to ganglion cells. Amacrine cells are often subclassified by neurotransmitter or neuromodulator or peptide (such as calretinin or calbindin) and interact with each other, with bipolar cells, and with photoreceptors. Bipolar cells are retinal interneurons that are named according to their morphology; bipolar cells receive input from the photoreceptors and sent the input to the ganglion cells. Horizontal cells modulate and transform visual information from large numbers of photoreceptors and have horizontal integration (whereas bipolar cells relay information radially through the retina).

Other retinal cells that may be present in the retinal cell cultures described herein include glial cells, such as Müller glial cells, and retinal pigmented epithelial cells (RPE). Glial cells surround nerve cell bodies and axons. The glial cells do not carry electrical impulses but contribute to maintenance of normal brain function. Müller glia, the predominant type of glial cell within the retina, provide structural support of the retina and are involved in the metabolism of the retina (e.g., contribute to regulation of ionic concentrations, degradation of neurotransmitters, and remove certain metabolites (see, e.g., Kljavin et al., J. Neurosci. 11:2985 (1991))). Müller's fibers (also known as sustentacular fibers of retina) are sustentacular neuroglial cells of the retina that run through the thickness of the retina from the internal limiting membrane to the bases of the rods and cones where they form a row of junctional complexes.

Retinal pigmented epithelial (RPE) cells form the outermost layer of the retina, nearest the blood vessel-enriched choroids. RPE cells are a type of phagocytic epithelial cell, functioning like macrophages, that lies below the photoreceptors of the eye. The dorsal surface of the RPE cell is closely apposed to the ends of the rods, and as discs are shed from the rod outer segment they are internalized and digested by RPE cells. RPE cells also produce, store, and transport a variety of factors that contribute to the normal function and survival of photoreceptors. Another function of RPE cells is to recycle vitamin A as it moves between photoreceptors and the RPE during light and dark adaptation.

Described herein is an exemplary long-term in vitro cell culture system permits and promotes the survival in the culture of mature retinal cells, including retinal neurons, for at least 2-4 weeks, over 2 months, or for as long as 6 months. The cell culture system is useful for identifying and characterizing retinoid compounds that are useful in the methods described herein for treating and/or preventing an ophthalmic disease or disorder or for preventing or inhibiting accumulation in the eye of lipofuscin and/or A2E. Retinal cells are isolated from non-embryonic, non-tumorigenic tissue and have not been immortalized by any method such as, for example, transformation or infection with an oncogenic virus. The cell culture system may comprise all the major retinal neuronal cell types (photoreceptors, bipolar cells, horizontal cells, amacrine cells, and ganglion cells), and also may include other mature retinal cells such as retinal pigmented epithelial cells and Müller glial cells.

### In Vivo and In Vitro Systems for Determining Effect of Retinylamine Compounds

In one embodiment, methods are provided for enhancing or prolonging neuronal cell survival, including retinal neuronal cell survival. Also provided herein are methods for inhibiting or preventing degeneration of a retinal cell, including a retinal neuronal cell (e.g., a photoreceptor cell, an amacrine cell, a horizontal cell, a bipolar cell, and a ganglion cell) and other mature retinal cells such as retinal pigmented epithelial cells and Müller glial cells. Such methods comprise administration of a retinylamine derivative compound as described herein. Such a compound is useful for enhancing or prolonging retinal cell survival, including photoreceptor cell survival, which can result in slowing or halting the progression of an ophthalmic disease or disorder or retinal injury, which are described herein.

The effect of a retinylamine compound on retinal cell survival may be determined by using cell culture models, animal models, and other methods that are described herein and practiced by persons skilled in the art. By way of example, and not limitation, such methods and assays include those described in Oglivie et al., Exp. Neurol. 161:675-856 (2000); U.S. Pat. No. 6,406,840; WO 01/81551; WO 98/12303; U.S. Patent Application No. 2002/0009713; WO 00/40699; U.S. Pat. No. 6,117,675; U.S. Pat. No. 5,736,516; WO 99/29279; WO 01/83714; WO 01/42784; U.S. Pat. No. 6,183,735; U.S. Pat. No. 6,090,624; WO 01/09327; U.S. Pat. No. 5,641,750; and U.S. patent application Ser. No. 10/903,880.

The lack of a good animal model has proved to be a major obstacle for developing new drugs to treat retinal diseases and disorders. For example, macula exist in primates (including humans) but not in rodents. A recently developed animal model may be useful for evaluating treatments for macular degeneration has been described by Ambati et al. (Nat. Med. 9:1390-97 (2003); Epub 2003 Oct. 19). This animal model is one of only a very few exemplary animal models presently available for evaluating a compound or any molecule for use in treating (including preventing) progression or development of a neurodegenerative disease, especially an ophthalmic disease. Accordingly, cell culture methods, such as the method described herein, is particularly useful for determining the effect of on retinal neuronal cell survival.

### Cell Culture System

An exemplary cell culture model is described herein and is described in detail in U.S. Patent Application Publication No. US 2005-0059148 (which is incorporated by reference in its entirety), which is useful for determining the capability of a retinylamine compound as described herein to enhance or prolong survival of neuronal cells, particularly retinal neuronal cells, and inhibit, prevent, slow, or retard degeneration of an eye, or the retina or retinal cells thereof, which molecules are useful for treating ophthalmic diseases and disorders.

The cell culture model comprises a long-term or extended culture of mature retinal cells, including retinal neuronal cells (e.g., photoreceptor cells, amacrine cells, ganglion cells, horizontal cells, and bipolar cells). The cell culture system and methods for producing the cell culture system provide extended culture of photoreceptor cells. The cell culture system may also comprise retinal pigmented epithelial (RPE) cells and Müller glial cells.

The retinal cell culture system may also comprise a cell stressor. The application or the presence of the stressor affects the mature retinal cells, including the retinal neuronal cells, in vitro in a manner that is useful for studying disease pathology that is observed in a retinal disease or disorder. The cell culture model described herein provides an in vitro neuronal cell culture system that will be useful in the identification and biological testing of a retinylamine compound that is suitable for treatment of neurological diseases or disorders in general, and for treatment of degenerative diseases of the eye and brain in particular. The ability to obtain primary cells from mature, fully-differentiated retinal cells, including retinal neurons for culture in vitro over an extended period of time in the presence of a stressor enables examination of cell-to-cell interactions, selection and analysis of neuroactive compounds and materials, use of a controlled cell culture system for in vivo CNS and ophthalmic tests, and analysis of the effects on single cells from a consistent retinal cell population.

The cell culture system and the retinal cell stress model comprise cultured mature retinal cells, retinal neurons, and a retinal cell stressor, which are particularly useful for screening and characterizing a retinylamine compound that are capable of inducing or stimulating regeneration of CNS tissue that has been damaged by disease. The cell culture system provides a mature retinal cell culture that is a mixture of mature retinal neuronal cells and non-neuronal retinal cells. The cell culture system may comprise all the major retinal neuronal cell types (photoreceptors, bipolar cells, horizontal cells, amacrine cells, and ganglion cells), and also includes other mature retinal cells such as RPE and Müller glial cells. By incorporating these different types of cells into the in vitro culture system, the system essentially resembles an “artificial organ” that is more akin to the natural in vivo state of the retina.

Viability of one or more of the mature retinal cell types is maintained for an extended period of time, for example, at least 4 weeks, 2 months (8 weeks), or at least 4-6 months, for at least 10%, 25%, 40%, 50%, 60%, 70%, 80%, or 90% of the mature retinal cells that are isolated (harvested) from retinal tissue and plated for tissue culture. Viability of the retinal cells may be determined according to methods described herein and known in the art. Retinal neuronal cells, similar to neuronal cells in general, are not actively dividing cells in vivo and thus cell division of retinal neuronal cells would not necessarily be indicative of viability. An advantage of the cell culture system is the ability to culture amacrine cells, photoreceptors, and associated ganglion projection neurons for extended periods of time, thereby providing an opportunity to determine the effectiveness of a retinylamine compound described herein for treatment of retinal disease.

The mature retinal cells and retinal neurons may be cultured in vitro for extended periods of time, longer than 2 days or 5 days, longer than 2 weeks, 3 weeks, or 4 weeks, and longer than 2 months (8 weeks), 3 months (12 weeks), and 4 months (16 weeks), and longer than 6 months, thus providing a long-term culture. At least 20-40%, at least 50%, at least 60%, at least 70%, at least 80%, or at least 90% of one or more of the mature retinal cell types remain viable in this long-term cell culture system. The biological source of the retinal cells or retinal tissue may be mammalian (e.g., human, non-human primate, ungulate, rodent, canine, porcine, bovine, or other mammalian source), avian, or from other genera Retinal cells including retinal neurons from post-natal non-human primates, post-natal pigs, or post-natal chickens may be used, but any adult or post-natal retinal tissue may be suitable for use in this retinal cell culture system.

The cell culture system provides for robust long-term survival of retinal cells without inclusion of cells derived from or isolated or purified from non-retinal tissue. The cell culture system comprises cells isolated solely from the retina of the eye and thus is substantially free of types of cells from other parts or regions of the eye that are separate from the retina, such as ciliary bodies and vitreous. A retinal cell culture that is substantially free of non-retinal cells contains retinal cells that comprise preferably at least 80-85% of the cell types in culture, preferably 90%-95%, or preferably 96%-100% of the cell types. Retinal cells in the cell culture system are viable and survive in the cell culture system without added purified (or isolated) glial cells or stem cells from a non-retinal source, or other non-retinal cells. The retinal cell culture system is prepared from isolated retinal tissue only, thereby rendering the cell culture system substantially free of non-retinal cells.

The in vitro retinal cell culture systems described herein may serve as physiological retinal models that can be used to characterize the physiology of the retina. This physiological retinal model may also be used as a broader general neurobiology model. A cell stressor may be included in the model cell culture system. A cell stressor, which as described herein is a retinal cell stressor, adversely affects the viability or reduces the viability of one or more of the different retinal cell types, including types of retinal neuronal cells, in the cell culture system. A person skilled in the art would readily appreciate and understand that as described herein a retinal cell that exhibits reduced viability means that the length of time that a retinal cell survives in the cell culture system is reduced or decreased (decreased lifespan) and/or that the retinal cell exhibits a decrease, inhibition, or adverse effect of a biological or biochemical function (e.g., decreased or abnormal metabolism; initiation of apoptosis; etc.) compared with a retinal cell cultured in an appropriate control cell system (e.g., the cell culture system described herein in the absence of the cell stressor). Reduced viability of a retinal cell may be indicated by cell death; an alteration or change in cell structure or morphology; induction and/or progression of apoptosis; initiation, enhancement, and/or acceleration of retinal neuronal cell neurodegeneration (or neuronal cell injury).

Methods and techniques for determining cell viability are described in detail herein and are those with which skilled artisans are familiar. These methods and techniques for determining cell viability may be used for monitoring the health and status of retinal cells in the cell culture system and for determining the capability of the retinylamine compounds described herein to alter (preferably increase, prolong, enhance, improve) retinal cell viability or retinal cell survival and to inhibit retinal cell degeneration.

The addition of a cell stressor to the cell culture system is useful for determining the capability of a retinylamine compound to abrogate, inhibit, eliminate, or lessen the effect of the stressor. The retinal neuronal cell culture system may include a cell stressor that is chemical (e.g., A2E, cigarette smoke concentrate); biological (for example, toxin exposure; beta-amyloid; lipopolysaccharides); or non-chemical, such as a physical stressor, environmental stressor, or a mechanical force (e.g., increased pressure or light exposure).

The retinal cell stressor model system may also include a cell stressor such as, but not limited to, a stressor that may be a risk factor in a disease or disorder or that may contribute to the development or progression of a disease or disorder, including but not limited to, light of varying wavelengths and intensities; cigarette smoke condensate exposure; glucose oxygen deprivation; oxidative stress (e.g., stress related to the presence of or exposure to hydrogen peroxide, nitroprusside, Zn++, or Fe++); increased pressure (e.g., atmospheric pressure or hydrostatic pressure), glutamate or glutamate agonist (e.g., N-methyl-D-aspartate (NMDA); alpha-amino-3-hydroxy-5-methylisoxazole-4-proprionate (AMPA); kainic acid; quisqualic acid; ibotenic acid; quinolinic acid; aspartate; trans-1-aminocyclopentyl-1,3-dicarboxylate (ACPD)); amino acids (e.g., aspartate, L-cysteine; beta-N-methylamine-L-alanine); heavy metals (such as lead); various toxins (for example, mitochondrial toxins (e.g., malonate, 3-nitroproprionic acid; rotenone, cyanide); MPTP (1-methyl-4-phenyl-1,2,3,6-tetrahydropyridine), which metabolizes to its active, toxic metabolite MPP+ (1-methyl-4-phenylpryidine)); 6-hydroxydopamine; alpha-synuclein; protein kinase C activators (e.g., phorbol myristate acetate); biogenic amino stimulants (for example, methamphetamine, MDMA (3-4 methylenedioxymethamphetamine)); or a combination of one or more stressors. Useful retinal cell stressors include those that mimic a neurodegenerative disease that affects any one or more of the mature retinal cells described herein. A chronic disease model is of particular importance because most neurodegenerative diseases are chronic. Through use of this in vitro cell culture system, the earliest events in long-term disease development processes may be identified because an extended period of time is available for cellular analysis.

A retinal cell stressor may alter (i.e., increase or decrease in a statistically significant manner) viability of retinal cells such as by altering survival of retinal cells, including retinal neuronal cells, or by altering neurodegeneration of retinal neuronal cells. Preferably, a retinal cell stressor adversely affects a retinal neuronal cell such that survival of a retinal neuronal cell is decreased or adversely affected (i.e., the length of time during which the cells are viable is decreased in the presence of the stressor) or neurodegeneration (or neuron cell injury) of the cell is increased or enhanced. The stressor may affect only a single retinal cell type in the retinal cell culture or the stressor may affect two, three, four, or more of the different cell types. For example, a stressor may alter viability and survival of photoreceptor cells but not affect all the other major cell types (e.g., ganglion cells, amacrine cells, horizontal cells, bipolar cells, RPE, and Müller glia). Stressors may shorten the survival time of a retinal cell (in vivo or in vitro), increase the rapidity or extent of neurodegeneration of a retinal cell, or in some other manner adversely affect the viability, morphology, maturity, or lifespan of the retinal cell.

The effect of a cell stressor on the viability of retinal cells in the cell culture system may be determined for one or more of the different retinal cell types. Determination of cell viability may include evaluating structure and/or a function of a retinal cell continually at intervals over a length of time or at a particular time point after the retinal cell culture is prepared. Viability or long term survival of one or more different retinal cell types or one or more different retinal neuronal cell types may be examined according to one or more biochemical or biological parameters that are indicative of reduced viability, such as apoptosis or a decrease in a metabolic function, prior to observation of a morphological or structural alteration.

A chemical, biological, or physical cell stressor may reduce viability of one or more of the retinal cell types present in the cell culture system when the stressor is added to the cell culture under conditions described herein for maintaining the long-term cell culture. Alternatively, one or more culture conditions may be adjusted so that the effect of the stressor on the retinal cells can be more readily observed. For example, the concentration or percent of fetal bovine serum may be reduced or eliminated from the cell culture when cells are exposed to a particular cell stressor. When a serum-free media is desired for a particular purpose, cells may be gradually weaned (i.e., the concentration of the serum is progressively and often systematically decreased) from an animal source of serum into a media that is free of serum or that contains a non-serum substitute. The decrease in serum concentration and the time period of culture at each decreased concentration of serum may be continually evaluated and adjusted to ensure that cell survival is maintained. When the retinal cell culture system is exposed to a cell stressor, the serum concentration may be adjusted concomitantly with the application of the stressor (which may also be titrated (if chemical or biological) or adjusted (if a physical stressor)) to achieve conditions such that the stress model is useful for evaluating the effect of the stressor on a retinal cell type and/or for identifying a retinylamine compound that inhibits, reduces, or abrogates the adverse effect(s) of a stressor on the retinal cell. Alternatively, retinal cells cultured in media containing serum at a particular concentration for maintenance of the cells may be abruptly exposed to media that does not contain any level of serum.

The retinal cell culture may be exposed to a cell stressor for a period of time that is determined to reduce the viability of one or more retinal cell types in the retinal cell culture system. The cells may be exposed to a cell stressor immediately upon plating of the retinal cells after isolation from retinal tissue. Alternatively, the retinal cell culture may be exposed to a stressor after the culture is established, or any time thereafter. When two or more cell stressors are included in the retinal cell culture system, each stressor may be added to the cell culture system concurrently and for the same length of time or may be added separately at different time points for the same length of time or for differing lengths of time during the culturing of the retinal cell system.

Viability of the retinal cells in the cell culture system may be determined by any one or more of several methods and techniques described herein and practiced by skilled artisans. The effect of a stressor may be determined by comparing structure or morphology of a retinal cell, including a retinal neuronal cell, in the cell culture system in the presence of the stressor with structure or morphology of the same cell type of the cell culture system in the absence of the stressor, and therefrom identifying a stressor that is capable of altering neurodegeneration of the neuronal cell. The effect of the stressor on viability can also be evaluated by methods known in the art and described herein, for example by comparing survival of a neuronal cell of the cell culture system in the presence of the stressor with survival of a neuronal cell of the cell culture system in the absence of the stressor.

Photoreceptors may be identified using antibodies that specifically bind to photoreceptor-specific proteins such as opsins, peripherins, and the like.

Photoreceptors in cell culture may also be identified as a morphologic subset of immunocytochemically labeled cells by using a pan-neuronal marker or may be identified morphologically in enhanced contrast images of live cultures. Outer segments can be detected morphologically as attachments to photoreceptors.

Retinal cells including photoreceptors can also be detected by functional analysis. For example, electrophysiology methods and techniques may be used for measuring the response of photoreceptors to light. Photoreceptors exhibit specific kinetics in a graded response to light. Calcium-sensitive dyes may also be used to detect graded responses to light within cultures containing active photoreceptors. For analyzing stress-inducing compounds or potential neurotherapeutics, retinal cell cultures can be processed for immunocytochemistry, and photoreceptors and/or other retinal cells can be counted manually or by computer software using photomicroscopy and imaging techniques. Other immunoassays known in the art (e.g., ELISA, immunoblotting, flow cytometry) may also be useful for identifying and characterizing the retinal cells and retinal neuronal cells of the cell culture model system described herein.

The retinal cell culture stress models may also be useful for identification of both direct and indirect pharmacologic agent effects by the bioactive agent of interest, such as a retinylamine compound. For example, a bioactive agent added to the cell culture system in the presence of one or more retinal cell stressors may stimulate one cell type in a manner that enhances or decreases the survival of other cell types. Cell/cell interactions and cell/extracellular component interactions may be important in understanding mechanisms of disease and drug function. For example, one neuronal cell type may secrete trophic factors that affect growth or survival of another neuronal cell type (see, e.g., WO 99/29279).

In another embodiment, a retinylamine derivative compound, is incorporated into screening assays comprising the retinal cell culture stress model system described herein to determine whether and/or to what level or degree the compound increases viability (i.e., increases in a statistically significant or biologically significant manner) of a plurality of retinal cells. A person skilled in the art would readily appreciate and understand that as described herein a retinal cell that exhibits increased viability means that the length of time that a retinal cell survives in the cell culture system is increased (increased lifespan) and/or that the retinal cell maintains a biological or biochemical function (normal metabolism and organelle function; lack of apoptosis; etc.) compared with a retinal cell cultured in an appropriate control cell system (e.g., the cell culture system described herein in the absence of the compound). Increased viability of a retinal cell may be indicated by delayed cell death or a reduced number of dead or dying cells; maintenance of structure and/or morphology; lack of or delayed initiation of apoptosis; delay, inhibition, slowed progression, and/or abrogation of retinal neuronal cell neurodegeneration or delaying or abrogating or preventing the effects of neuronal cell injury. Methods and techniques for determining viability of a retinal cell and thus whether a retinal cell exhibits increased viability are described in greater detail herein and are known to persons skilled in the art.

In certain embodiments, a method is provided for determining whether a retinylamine compound, enhances survival of photoreceptor cells. One method comprises contacting a retinal cell culture system as described herein with the agent under conditions and for a time sufficient to permit interaction between the retinal neuronal cells and the compound. Enhanced survival (prolonged survival) may be measured according to methods described herein and known in the art, including detecting expression of rhodopsin. Rhodopsin, which is composed of the protein opsin and retinal (a vitamin A form), is located in the membrane of the photoreceptor cell in the retina of the eye and catalyzes the only light sensitive step in vision. The 11-cis-retinal chromophore lies in a pocket of the protein and is isomerized to all-trans retinal when light is absorbed. The isomerization of retinal leads to a change of the shape of rhodopsin, which triggers a cascade of reactions that lead to a nerve impulse that is transmitted to the brain by the optical nerve.

The capability of a retinylamine compound, to increase retinal cell viability and/or to enhance, promote, or prolong cell survival (that is, to extend the time period in which retinal neuronal cells are viable), and/or impair, inhibit, or impede neurodegeneration as a direct or indirect result of the herein described stress may be determined by any one of several methods known to those skilled in the art. For example, changes in cell morphology in the absence and presence of the compound, may be determined by visual inspection such as by light microscopy, confocal microscopy, or other microscopy methods known in the art. Survival of cells can also be determined by counting viable and/or nonviable cells, for instance.

Immunochemical or immunohistological techniques (such as fixed cell staining or flow cytometry) may be used to identify and evaluate cytoskeletal structure (e.g. by using antibodies specific for cytoskeletal proteins such as glial fibrillary acidic protein, fibronectin, actin, vimentin, tubulin, or the like) or to evaluate expression of cell markers as described herein. The effect of a retinylamine compound on cell integrity, morphology, and/or survival may also be determined by measuring the phosphorylation state of neuronal cell polypeptides, for example, cytoskeletal polypeptides (see, e.g., Sharma et al., J. Biol. Chem. 274:9600-06 (1999); Li et al., J. Neurosci. 20:6055-62 (2000)). Cell survival or, alternatively cell death, may also be determined according to methods described herein and known in the art for measuring apoptosis (for example, annexin V binding, DNA fragmentation assays, caspase activation, marker analysis, e.g., poly(ADP-ribose) polymerase (PARP), etc.).

Enhanced survival (or prolonged or extended survival) of one or more retinal cell types in the presence of a retinylamine compound indicates that the compound may be an effective agent for treatment of a neurodegenerative disease, particularly a retinal disease or disorder. Cell survival and enhanced cell survival may be determined according to methods described herein and known to a skilled artisan including viability assays and assays for detecting expression of retinal cell marker proteins. For determining enhanced survival of photoreceptor cells, opsins may be detected, for instance, including the protein rhodopsin that is expressed by rods.

In another embodiment, the subject is being treated for Stargardt's disease or Stargardt's macular degeneration. In Stargardt's disease, which is associated with mutations in the ABCA4 (also called ABCR) transporter, the accumulation of all-trans-retinal has been proposed to be responsible for the formation of a lipofuscin pigment, A2E, which is toxic towards retinal cells and causes retinal degeneration and consequently loss of vision.

In yet another embodiment, the subject is being treated for age-related macular degeneration (AMD). In various embodiments, AMD can be wet or dry form. In AMD, vision loss occurs when complications late in the disease either cause new blood vessels to grow under the retina or the retina atrophies. Without intending to be bound by any particular theory, the accumulation of all-trans-retinal has been proposed to be responsible for the formation of a lipofuscin pigment, N-retinylidene-N-retinylethanolamine (A2E), which is toxic towards retinal cells and causes retinal degeneration and consequently loss of vision.

In the vertebrate eye, for example, a mammalian eye, the formation of A2E is a light-dependent process and its accumulation leads to a number of negative effects in the eye. These include destabilization of retinal pigment epithelium (RPE) membranes, sensitization of cells to blue-light damage, and impaired degradation of phospholipids. Products of A2E oxidation by molecular oxygen (oxiranes) were even shown to induce DNA damage in cultured RPE cells. All these factors lead to a gradual decrease in visual acuity and eventually to vision loss. If it were possible to reduce the formation of retinals during vision processes, it would lead to decreased amounts of A2E in the eye. This would delay the aging of the RPE and retina and would slow down or prevent vision loss. Treating patients with 11-cis-retinylamine can prevent or slow the formation of A2E and can have protective properties for the retina.

### Treatment of Neurodegenerative Diseases

In another embodiment, methods are provided for treating and/or preventing neurodegenerative diseases and disorders, particularly neurodegenerative retinal diseases and ophthalmic diseases as described herein. A subject in need of such treatment may be a human or non-human primate or other animal who has developed symptoms of a neurodegenerative retinal disease or who is at risk for developing a neurodegenerative retinal disease. As described herein a method is provided for treating (which includes preventing or prophylaxis) an ophthalmic disease or disorder by administrating to a subject in need thereof a composition comprising a pharmaceutically acceptable carrier and a retinylamine compound (e.g., a compound having the structure of any one of formulas I-V and substructures thereof). As described herein, a method is provided for enhancing or prolonging survival of neuronal cells such as retinal neuronal cells, including photoreceptor cells, and/or inhibiting degeneration (prolonging or enhancing survival or viability) of retinal cells, including retinal neuronal cells, by administering the compositions described herein comprising a retinylamine compound.

A neurodegenerative retinal disease or disorder for which the compounds and methods described herein may be used for treating, curing, preventing, ameliorating the symptoms of, or slowing, inhibiting, or stopping the progression of, is a disease or disorder that leads to or is characterized by retinal neuronal cell loss, which is the cause of visual impairment. Such a disease or disorder includes but is not limited to diabetic retinopathy, diabetic maculopathy, diabetic macular edema, retinal ischemia, ischemia-reperfusion related retinal injury, and metabolic optic neuropathy. Other ophthalmic diseases and disorders that may be treated using the methods and compositions described herein include macular degeneration (including dry form and wet form of macular degeneration), glaucoma, retinal detachment, retinal blood vessel (artery or vein) occlusion, hemorrhagic retinopathy, retinitis pigmentosa, retinopathy of prematurity, an inflammatory retinal disease, proliferative vitreoretinopathy, retinal dystrophy, hereditary optic neuropathy, Stargardt's macular dystrophy, Sorsby's fundus dystrophy, Best disease, uveitis, a retinal injury, optical neuropathy, and retinal disorders associated with other neurodegenerative diseases such as Alzheimer's disease, multiple sclerosis, Parkinson's disease or other neurodegenerative diseases that affect brain cells, a retinal disorder associated with viral infection, or other conditions such as AIDS. A retinal disorder also includes light damage to the retina that is related to increased light exposure (i.e., overexposure to light), for example, accidental strong or intense light exposure during surgery; strong, intense, or prolonged sunlight exposure, such as at a desert or snow covered terrain; during combat, for example, when observing an explosion or from a laser device, and the like.

Macular degeneration as described herein is a disorder that affects the macula (central region of the retina) and results in the decline and loss of central vision. Age-related macular degeneration occurs typically in individuals over the age of 55 years. The etiology of age-related macular degeneration may include both an environmental influence and a genetic component (see, e.g., Lyengar et al., Am. J. Hum. Genet. 74:20-39 (2004) (Epub 2003 Dec. 19); Kenealy et al., Mol. Vis. 10:57-61 (2004); Gorin et al., Mol. Vis. 5:29 (1999)). More rarely, macular degeneration occurs in younger individuals, including children and infants, and generally the disorder results from a genetic mutation. Types of juvenile macular degeneration include Stargardt's disease (see, e.g., Glazer et al., Opthalmol. Clin. North Am. 15:93-100, viii (2002); Weng et al., Cell 98:13-23 (1999)); Best's vitelliform macular dystrophy (see, e.g., Kramer et al., Hum. Mutat. 22:418 (2003); Sun et al., Proc. Natl. Acad. Sci. USA 99:4008-13 (2002)), Doyne's honeycomb retinal dystrophy (see, e.g., Kermani et al., Hum. Genet. 104:77-82 (1999)); Sorsby's fundus dystrophy, Malattia Levintinese, fundus flavimaculatus, and autosomal dominant hemorrhagic macular dystrophy (see also Seddon et al., Opthalmology 108:2060-67 (2001); Yates et al., J. Med. Genet. 37:83-7 (2000); Jaakson et al., Hum. Mutat. 22:395-403 (2003)).

Stargardt's macular degeneration, a recessive inherited disease, is an inherited blinding disease of children. The primary pathologic defect in Stargardt's disease is also an accumulation of toxic lipofuscin pigments such as A2E in cells of the retinal pigment epithelium (RPE). This accumulation appears to be responsible for the photoreceptor death and severe visual loss found in Stargardt's patients. Retinylamine can slow the synthesis of 11-cis-retinaldehyde (11cRAL) and regeneration of −5-rhodopsin by inhibiting isomerase in the visual cycle. Light activation of rhodopsin results in its release of all-trans-retinal, which constitutes the first reactant in A2E biosynthesis. Treatment with retinylamine can inhibit lipofuscin accumulation and thus delay the onset of visual loss in Stargardt's and AMD patients without toxic effects that would preclude treatment with a retinylamine compound. The compounds described herein may be used for effective treatment of other forms of retinal or macular degeneration associated with lipofuscin accumulation.

Administration of a synthetic retinylamine derivative compound described herein to a subject may prevent formation of the lipofuscin pigment, A2E, which is toxic towards retinal cells and causes retinal degeneration. In certain embodiments, administration of a retinylamine compound may lessen the production of waste products, e.g., lipofuscin pigment, A2E, and reduce or slow vision loss (e.g., choroidal neovascularization and/or chorioretinal atrophy). In previous studies, with 13-cis-retinoic acid (Accutane® or Isotretinoin), a drug commonly used for the treatment of acne and an inhibitor of 11-cis-retinol dehydrogenase, has been administered to patients to prevent A2E accumulation in the RPE. However, a major drawback in this proposed treatment is that 13-cis-retinoic acid can easily isomerize to all-trans-retinoic acid. All-trans-retinoic acid is a very potent teratogenic compound that causes adverse effects cell proliferation and development. Retinoic acid also accumulates in the liver and may be a contributing factor in liver diseases.

In yet other aspects, a retinylamine compound is administered to a subject such as a human with a mutation in the ABCA4 transporter in the eye. The retinylamine compound can also be administered to an aging subject. As used herein, an aging human subject is typically at least 45, or at least 50, or at least 60, or at least 65 years old. In Stargardt's disease, associated with mutations in the ABCA4 transporter, the accumulation of all-trans-retinal has been proposed to be responsible for the formation of a lipofuscin pigment, A2E, which is toxic towards retinal cells and causes retinal degeneration and consequently loss of vision. Without wishing to be bound by theory, a retinylamine compound described herein can be a strong inhibitor of the isomerohydrolase protein involved in the visual cycle. Treating a subject with a retinylamine derivative, e.g., 1-cis-retinylamine can prevent or slow the formation of A2E and can have protective properties for normal vision. Such treatment may also decrease or inhibit or suppress production or accumulation of other retinoid related toxic by-products, for example, fatty exudates that may accumulate in patients who have diabetes.

As used herein, a patient (or subject) may be any mammal, including a human, that may have or be afflicted with a neurodegenerative disease or condition, including an ophthalmic disease or disorder, or that may be free of detectable disease. Accordingly, the treatment may be administered to a subject who has an existing disease, or the treatment may be prophylactic, administered to a subject who is at risk for developing the disease or condition. Treating or treatment by administering an effective amount of at least one of the retinylamine derivative compounds described herein refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being.

The treatment or amelioration of symptoms can be based on objective or subjective parameters; including the results of a physical examination. Accordingly, the term “treating” includes the administration of the compounds or agents described herein to treat pain, hyperalgesia, allodynia, or nociceptive events and to prevent or delay, to alleviate, or to arrest or inhibit development of the symptoms or conditions associated with pain, hyperalgesia, allodynia, nociceptive events, or other disorders. The term “therapeutic effect” refers to the reduction, elimination, or prevention of the disease, symptoms of the disease, or sequelae of the disease in the subject. Treatment also includes restoring or improving retinal neuronal cell functions (including photoreceptor function) in a vertebrate visual system, for example, such as visual acuity and visual field testing etc., as measured over time (e.g., as measured in weeks or months). Treatment also includes stabilizing disease progression (i.e., slowing, minimizing, or halting the progression of an ophthalmic disease and associated symptoms) and minimizing additional degeneration of a vertebrate visual system. Treatment also includes prophylaxis and refers to the administration of a retinylamine compound to a subject in need thereof to prevent degeneration or further degeneration or deterioration or further deterioration of the vertebrate visual system of the subject and to prevent or inhibit development of the disease and/or related symptoms and sequelae.

A subject or patient refers to any vertebrate or mammalian patient or subject to whom the compositions described herein can be administered. The term “vertebrate” or “mammal” includes humans and non-human primates, as well as experimental animals such as rabbits, rats, and mice, and other animals, such as domestic pets and zoo animals. Subjects in need of treatment using the methods described herein may be identified according to accepted screening methods in the medical art that are employed to determine risk factors or symptoms associated with an ophthalmic disease or condition described herein or to determine the status of an existing ophthalmic disease or condition in a subject. These and other routine methods allow the clinician to select patients in need of therapy that includes the methods and compositions described herein.

The retinylamine derivative compounds are preferably combined with a pharmaceutical carrier (i.e., a pharmaceutically acceptable excipient, diluent, etc., which is a non-toxic material that does not interfere with the activity of the active ingredient) selected on the basis of the chosen route of administration and standard pharmaceutical practice as described, for example, in Remington's Pharmaceutical Sciences (Mack Pub. Co., Easton, Pa., 1980), the disclosure of which is hereby incorporated herein by reference, in its entirety.

Although a retinylamine derivative compound may be administered as a pure chemical, preferably the active ingredient is administered as a pharmaceutical composition. Accordingly, provided herein is a pharmaceutical composition comprising one or more retinylamine compounds, such as a positively charged retinoid compound, or a stereoisomer, prodrug, pharmaceutically or opthalmologically acceptable salt, hydrate, solvate, acid salt hydrate, N-oxide or isomorphic crystalline form thereof, together with one or more pharmaceutically acceptable carriers therefore and, optionally, other therapeutic and/or prophylactic ingredients. The carrier(s) must be acceptable in the sense of being compatible with the other ingredients of the composition and not deleterious to the recipient thereof. A pharmaceutically acceptable or suitable composition includes an opthalmologically suitable or acceptable composition.

A pharmaceutical composition (e.g., for oral administration or delivery by injection or for application as an eye drop) may be in the form of a liquid. A liquid pharmaceutical composition may include, for example, one or more of the following: sterile diluents such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition or a composition that is delivered ocularly is preferably sterile.

A retinylamine derivative compound can be administered to human or other nonhuman vertebrates. In certain embodiments, the compound is substantially pure, in that is contains less than about 5% or less than about 1%, or less than about 0.1%, of other retinoids. In other embodiments, a combination of retinylamine compounds can be administered.

A retinylamine derivative compound can be delivered to the eye by any suitable means, including, for example, oral or local administration. Modes of local administration can include, for example, eye drops, intraocular injection or periocular injection. Periocular injection typically involves injection of the synthetic retinylamine derivative into the conjunctiva or to the tennon (the fibrous tissue overlying the eye). Intraocular injection typically involves injection of the synthetic retinylamine derivative into the vitreous. In certain embodiments, the administration is non-invasive, such as by eye drops or oral dosage form.

A retinylamine derivative compound can be formulated for administration using pharmaceutically acceptable (suitable) carriers or vehicles as well as techniques routinely used in the art. A pharmaceutically acceptable or suitable carrier includes an opthalmologically suitable or acceptable carrier. A vehicle is selected according to the solubility of the retinylamine compound. Suitable opthalmological compositions include those that are administrable locally to the eye, such as by eye drops, injection or the like. In the case of eye drops, the formulation can also optionally include, for example, opthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to opthalmologic formulations, such as within the range of about pH 4 to 8.

For injection, the retinylamine derivative compound can be provided in an injection grade saline solution, in the form of an injectable liposome solution, or the like. Intraocular and periocular injections are known to those skilled in the art and are described in numerous publications including, for example, Spaeth, Ed., Ophthalmic Surgery: Principles of Practice, W. B. Sanders Co., Philadelphia, Pa., 85-87, 1990.

Suitable oral dosage forms include, for example, tablets, pills, sachets, or capsules of hard or soft gelatin, methylcellulose or of another suitable material easily dissolved in the digestive tract. Suitable nontoxic solid carriers can be used which include, for example, pharmaceutical grades of mannitol, lactose, starch, magnesium stearate, sodium saccharin, talcum, cellulose, glucose, sucrose, magnesium carbonate, and the like. (See, e.g., Gennaro, Ed., Remington “Pharmaceutical Sciences”, 17 Ed., Mack Publishing Co., Easton, Pa., 1985.

The retinylamine derivative compounds described herein may be formulated for sustained or slow release. Such compositions may generally be prepared using well known technology and administered by, for example, oral, periocular, intraocular, rectal or subcutaneous implantation, or by implantation at the desired target site. Sustained-release formulations may contain an agent dispersed in a carrier matrix and/or contained within a reservoir surrounded by a rate controlling membrane. Excipients and carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Systemic drug absorption of a drug or composition administered via an ocular route is understood by persons skilled in the art (see, e.g., Lee et al., Int. J. Pharm. 233:1-18 (2002)). In one embodiment, a retinylamine compound is delivered by a topical ocular delivery method (see, e.g., Curr. Drug Metab. 4:213-22 (2003)). The composition may be in the form of an eye drop, salve, or ointment or the like, such as, aqueous eye drops, aqueous ophthalmic suspensions, non-aqueous eye drops, and non-aqueous ophthalmic suspensions, gels, ophthalmic ointments, etc. For preparing a gel, for example, carboxyvinyl polymer, methyl cellulose, sodium alginate, hydroxypropyl cellulose, ethylene maleic anhydride polymer and the like can be used. The dose of the composition comprising at least one of the retinylamine derivative compounds described herein may differ, depending upon the patient's (e.g., human) condition, that is, stage of the disease, general health status, age, and other factors that a person skilled in the medical art will use to determine dose. When the composition is used as eye drops, for example, one to several drops per unit dose, preferably 1 or 2 drops (about 50 μl per 1 drop), may be applied about 1 to about 6 times daily.

Pharmaceutical compositions may be administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with neurodegeneration of retinal neuronal cells. Optimal doses may generally be determined using experimental models and/or clinical trials. The optimal dose may depend upon the body mass, weight, or blood volume of the patient.

The doses of the retinylamine compounds can be suitably selected depending on the clinical status, condition and age of the subject, dosage form and the like. In the case of eye drops, a synthetic retinylamine derivative can be administered, for example, from about 0.01 mg, about 0.1 mg, or about 1 mg, to about 25 mg, to about 50 mg, to about 90 mg per single dose. Eye drops can be administered one or more times per day, as needed. In the case of injections, suitable doses can be, for example, about 0.0001 mg, about 0.001 mg, about 0.01 mg, or about 0.1 mg to about 10 mg, to about 25 mg, to about 50 mg, or to about 90 mg of the synthetic retinylamine derivative, one to four times per week. In other embodiments, about 1.0 to about 30 mg of synthetic retinylamine derivative can be administered one to three times per week.

Oral doses can typically range from about 1.0 to about 1000 mg, one to four times, or more, per day. An exemplary dosing range for oral administration is from about 10 to about 250 mg one to three times per day.

Other embodiments and uses will be apparent to one skilled in the art in light of the present disclosures. The following examples are provided merely as illustrative of various embodiments and shall not be construed to limit the invention in any way.

### EXAMPLES

### Example 1

### Experimental Procedures

Materials—Fresh bovine eyes are obtained from a local slaughterhouse (Schenk Packing Co., Inc., Stanwood, Wash.). Preparation of bovine RPE microsornes is performed according to previously described methods (Stecher et al., J Biol Chem 274:8577-85, 1999; see also Golczak et al., supra). All chemicals are purchased from Sigma-Aldrich (St. Louis, Mo.). 11-cis-Retinal is obtained from Dr. Rosalie Crouch (Medical University of South Carolina, Charleston, S.C.). Alternatively, 11-cis-Retinal may be purchased or synthesized as described herein.

Retinoid preparations—All-trans-retinol is obtained by reduction of all-trans-retinal with an excess of NaBH₄ in EtOH at 0° C. and purified by normal phase HPLC (Beckman Ultrasphere Si 5μ 4.5×250 mm, 10% EtOAc/hexane; detection at 325 nm). Purified all-trans-retinol is dried under a stream of argon and dissolved in DMF to a final concentration of 3 mM and stored at −80° C. Retinoid concentrations in EtOH are determined spectrophotometrically. Absorption coefficients for Ret-NH₂s (retinylamines) are assumed to be equal to those of retinol isomers (Hubbard et al., Methods Enzymol. 18:615-53 (1971); Robeson et al., J. Am. Chem. Soc. 77:4111-19 (1955)).

Chemical synthesis—Ret-NH₂ is obtained by a previously described method (Yang et al., Proc. Natl. Acad. Sci. USA 94:13559-64 (1997); see also Golczak et al., supra) with some modifications. The corresponding isomer of retinal is dissolved in EtOH and reacted with a 5-fold excess of 7 N NH₃ in MeOH for 1 hour at room temperature to form retinylimine. Then retinylimine is reduced to Ret-NH₂ with a 5-fold excess of NaBH₄. The reaction progress is followed spectrophotometrically. After 1 hour at 0° C., water is added and Ret-NH₂ is extracted twice with hexane. Combined hexane extracts are washed with water and brine, layers are separated, and the organic phase is loaded on a silica gel. The column is washed with hexane, then with 1:1 EtOAc/hexane. Ret-NH₂ is eluted with EtOAc with an addition of 10% 7 N NH₃/MeOH. The typical yield is 30% of pure Ret-NH₂. Prior to in vitro experiments, Ret-NH₂ is further purified using normal phase columns by elution with EtOAc/7 N NH₃ in MeOH (99:0.5).

Synthesis and HPLC separation of retinylamine isomers is performed as follows (see, e.g., Golczak et al., Proc. Natl. Acad. Sci. USA 102:8162-67 (2005)). Ret-NH₂ is synthesized by oxidation of retinol to retinal with MnO₂ (shift of A_{λmax} from 325 to 383 nm). The oxidation product is further reacted with NH₃ in order to produce Ret-NH₂ (progress of the reaction is concomitant with blue shift of the absorbance maximum as well as significant red shift upon acidification). Retinylimine is reduced by NaBH₄ to Ret-NH₂ (A_{λmax}=325 nm).

N-Substituted all-trans-Ret-NH₂s is prepared as described above, but instead of NH₃, an excess of the corresponding alkylamine is added to the solution of all-trans-retinal in EtOH. N-Alkyl-Ret-NH₂s are purified on an HPLC column using the conditions described above.

Hydroxylamine derivatives are prepared by the reaction of retinal with the corresponding hydroxylamines in EtOH. All-trans-retinal oximes are extracted with hexane, dried, redissolved in EtOH:MeOH (1:1) with an addition of acetic acid (10% v/v), and reduced with NaBH₃CN. MS analyses of synthesized retinoids are performed using a Kratos profile HV-3 direct probe mass spectrometer.

Retinyl amides are prepared by the reaction between all-trans-retinylamine and an excess of either acetic anhydride or palmitoyl chloride in anhydrous dichloromethane in the presence of N,N-dimethylaminopyridine at 0° C. for 30 min. After the reaction is complete, water is added and the product is extracted with hexane. The hexane layer is washed twice with water, dried with anhydrous magnesium sulfate, filtered, and evaporated. Mass analyses of synthesized retinoids are performed using a Kratos profile HV-3 direct probe mass spectrometer.

### Example 2

### Isomerase and LRAT Reaction

The capability of several retinylamine compounds to inhibit the activity of visual cycle trans-cis isomerohydrolase (isomerase) was determined.

Isomerase and LRAT reaction—The isomerase reaction was performed essentially as described previously (Stecher et al., J Biol Chem 274:8577-85 (1999); see also Golczak et al., supra). Bovine Retinal Pigment Epithelium (RPE) microsome membranes were the source of visual cycle trans-cis isomerohydrolase (isomerase).

RPE microsome membrane extracts may be purchased or prepared according to methods practiced in the art and stored at −80° C. Crude RPE microsome extracts were thawed in a 37° C. water bath, and then immediately placed on ice. 50 ml crude RPE microsomes were placed into a 50 ml Teflon-glass homogenizer (Fisher Scientific, catalog no. 0841416M) on ice, powered by a hand-held DeWalt drill, and homogenized ten times up and down on ice under maximum speed. This process was repeated until the crude RPE microsome solution was homogenized. The homogenate was then subjected to centrifugation (50.2 Ti rotor (Beckman, Fullerton, Calif.), 13,000 RPM; 15360 Rcf) for 15 minutes at 4° C. The supernatant was collected and subjected to centrifugation a5 42,000 RPM (160,000 Rcf, 50.2 Ti rotor) for 1 hour at 4° C. The supernatant was removed, and the pellets were suspended in 12 ml (final volume) cold 10 mM MOPS buffer, pH 7.0. The resuspended RPE membranes in 5 ml aliquots were homogenized in a glass-to-glass homogenizer (Fisher Scientific, catalog no. K885500-0021) to high homogeneity. Protein concentration was quantified using the BCA protein assay according to the manufacturer's protocol (Pierce, Rockford, Ill.; catalog no. 23227). The homogenized RPE preparations were stored at −80° C.

Recombinant human apo cellular retinaldehyde-binding protein (CRALBP) was cloned and expressed according to standard methods in the molecular biology art (see Crabb et al., Protein Science 7:746-57 (1998); Crabb et al., J. Biol. Chem. 263:18688-92 (1988)). Briefly, total RNA was prepared from confluent ARPE19 cells (American Type Culture Collection, Manassas, Va.), cDNA was synthesized using an oligo(dT)₁₂₋₁₈ primer, and then DNA encoding CRALBP was amplified by two sequential polymerase chain reactions (see Crabb et al., J. Biol. Chem. 263:18688-92 (1988); Intres, et al., J. Biol. Chem. 269:25411-18 (1994); GenBank Accession No. L34219.1). The PCR product was sub-cloned into pTrcHis2-TOPO TA vector according to the manufacturer's protocol (Invitrogen Inc., Carlsbad, Calif.; catalog no. K4400-01), and then the sequence was confirmed according to standard nucleotide sequencing techniques. Recombinant 6×His-tagged human CRALBP was expressed in One Shot TOP 10 chemically competent E. coli cells (Invitrogen), and the recombinant polypeptide was isolated from E. coli cell lysates by nickel affinity chromatography using Ni Sepharose XKI 6-20 columns for HPLC (Amersham Bioscience, Pittsburgh, Pa.; catalog no. 17-5268-02). The purified 6×His-tagged human CRALBP was dialyzed against 10 mM bis-tris-Propane (BTP) and analyzed by SDS-PAGE. The molecular weight of the recombinant human CRALBP was approximately 39 kDal.

The isomerase assay was performed in 10 mM BTP buffer, pH 7.5, 1% BSA, containing 1 mM ATP and 6 μM apo-CRALBP (cellular retinaldehyde-binding protein). To investigate inhibition properties of retinylamine derivative compounds, RPE microsomes were preincubated for 5 min in 37° C. with a compound in 10 mM BTP buffer, pH 7.5, 1% BSA, and 1 mM ATP prior to addition of apo-CRALBP and 10 μM all-trans-retinol. Retinylamine derivative compounds were delivered to the reaction mixture in 2 μl ethanol. If the compounds were not soluble in ethanol, DMF was added until the compound was in solution. The same volume of ethanol and/or DMF was added to the control reaction (absence of test compound). Bovine REP microsomes (see above) were then added, and the mixtures transferred to 37° C. to initiate the reaction (total volume=200 μl). The reactions were stopped after 30 minutes by adding methanol (300 μl). Heptane was added (300 μl) and mixed into the reaction mixture by pipetting. Retinoid was extracted by agitating the reaction mixtures, followed by centrifugation in a microcentrifuge. The upper organic phase was transferred to HPLC vials and then analyzed by HPLC using an Agilent 1100 HPLC system with normal phase column: SILICA (Agilent Technologies, dp 5μ, 4.6 mmX, 25CM). The solvent components were 20% of 2% isopropanol in ethyl acetate and 80% of 100% Hexane. Each experiment was performed three times in duplicate. Inhibition of isomerase activity (IC₅₀) was determined for each compound and is presented in Table 1 below.

TABLE 1
INHIBITION OF ISOMERASE BY RETINYLAMINE DERIVATIVE COMPOUNDS

| Compound | Structure | IC50 (μM) |
|---|---|---|
| Cmpd 1 | 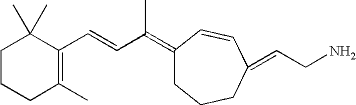 | 3.1 |
| Cmpd 2 | 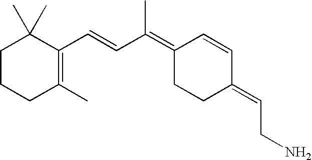 | 0.55 |
| Cmpd 3 | 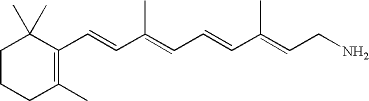 | 0.7 |
| Cmpd 4 | 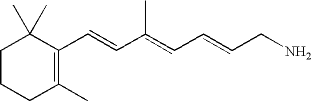 | 5.8 |
| Cmpd 5 | 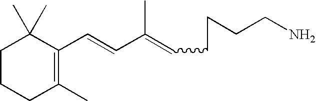 | 1.7 |
| Cmpd 6 | 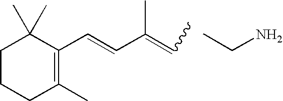 | 7.7 |
| Cmpd 7 | 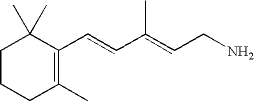 | 8.4 |
| Cmpd 8 | 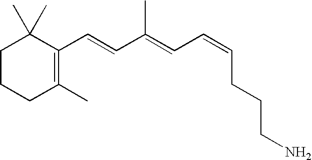 | 0.6 |
| Cmpd 9 | 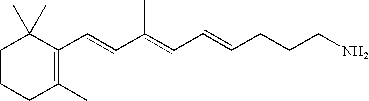 | 6 |
| Cmpd 10 | 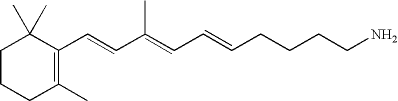 | 14 |
| Cmpd 11 | 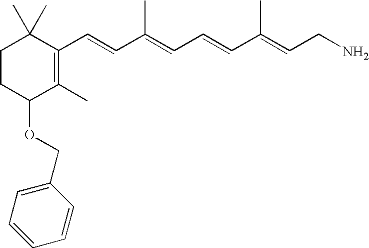 | 17 |
| Cmpd 12 | 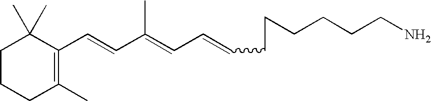 | 25 |
| Cmpd 13 | 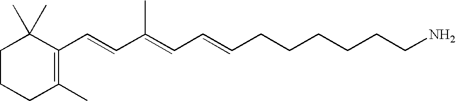 | 200 |
| Cmpd 14 | 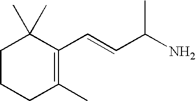 | 80 |

### Example 3

### In Vivo Murine Isomerase Assay

The capability of the retinylamine derivatives to inhibit isomerase is determined by an in vivo murine isomerase assay. Brief exposure of the eye to intense light (“photobleaching” of the visual pigment or simply “bleaching”) is known to photo-isomerize almost all 11-cis-retinal in the retina. The recovery of 11-cis-retinal after bleaching can be used to estimate the activity of isomerase in vivo. The regeneration of 11-cis-retinal after the photobleach (3,000 lux of white light for 10 minutes) in CD-1 (albino) mice that have been gavaged orally with compounds dissolved in corn oil containing 10% ethanol is assessed at various time intervals.

### Eye Retinoid Extraction

All steps are performed in darkness with minimal redlight illumination (low light darkroom lights and redfiltered flashlights for spot illumination as needed) (see, e.g., Maeda et al., J. Neurochem 85:944-956, 2003; Van Hooser et al., J Biol Chem 277:19173-82, 2002). Mice (6 weeks old) are sacrificed and the eyes are immediately removed and placed in liquid nitrogen. The eyes are then homogenized in a glass/glass homogenizer (Kontes Glass Co., homogenizer & pestle 21) containing 1 ml retinoid analysis buffer (50 mM MOPS, 10 mM NH₂OH, 50% EtOH, pH 7.0. The eyes are homogenized until no visible tissue remains (approximately 3 minutes). The samples are incubated 20 minutes at room temperature (including homogenizing) and then placed on ice. One ml cold EtOH is added to the homogenate to rinse the pestle, and the homogenate mixture is transferred to 7 ml glass screwtop tubes on ice. The homogenizer is rinsed with 7 ml hexane, which is added to the 7 ml tubes on ice.

The homogenate is mixed by vortexing at high speed for 1 minute. The phases are separated by centrifugation (5 minutes at 4000 rpm, 4° C.). The upper phase is collected and transferred to a clean glass test tube, taking care to avoid disturbing the interface by leaving approximately 0.2 ml of upper phase in the tube. The tubes with the collected upper phase are placed in a heating block at 25° C. and dried under a stream of Argon (˜30 minutes). The lower phase is again extracted by adding 4 ml hexane, vortexing, and separating the phases by centrifugation. The upper phase is collected as described above and pooled into the drying tubes. The dried samples are solubilized in 300 μl Hexane (Fisher Optima grade) and vortexed lightly. The samples are transferred to clean 300 μl glass inserts in HPLC vials using glass pipette and the vials are crimped shut tightly.

The samples are analyzed by HPLC(HP 1100 series or Agilent 1100 series, Agilent Technologies) on a Beckman Ultraspere Si column (5μ particle diameter, 4.6 mm ID×25 cm length; Part # 235341). Run parameters are as follows: flow: 1.4 ml/minute, 10% Ethylacetate+90% Hexane; detection at 325 nm (max absorption of Retinol).

Electroretinograms (ERGs)—Mice are prepared and ERG recording is performed as previously published (Haeseleer et al., Nat Neurosci 7:1079-87, 2004). Single flash stimuli had a range of intensities (−3.7-2.8 log cd·s·m⁻²). Typically, three to four animals are used for the recording of each point in all conditions. Statistical analysis is carried out using the one-way ANOVA test.

See also Deigner et al., Science, 244: 968-971, 1989; Gollapalli et al., Biochim Biophys Acta. 1651: 93-101, 2003; Parish, et al., Proc. Natl. Acad. Sci. USA, 14609-14613, 1998; Radu, et al., Proc Natl Acad Sci USA, 101: 5928-5933, 2004.

### Example 4

### Preparation of Retinal Neuronal Cell Culture System

This Example describes methods for preparing a long-term culture of retinal neuronal cells.

All compounds and reagents are obtained from Sigma Aldrich Chemical Corporation (St. Louis, Mo.) except as noted.

### Retinal Neuronal Cell Culture

Porcine eyes are obtained from Kapowsin Meats, Inc. (Graham, Wash.). Eyes are enucleated, and muscle and tissue are cleaned away from the orbit. Eyes are cut in half along their equator and the neural retina is dissected from the anterior part of the eye in buffered saline solution, according to standard methods known in the art. Briefly, the retina, ciliary body, and vitreous are dissected away from the anterior half of the eye in one piece, and the retina is gently detached from the clear vitreous. Each retina is dissociated with papain (Worthington Biochemical Corporation, Lakewood, N.J.), followed by inactivation with fetal bovine serum (FBS) and addition of 134 Kunitz units/ml of DNaseI. The enzymatically dissociated cells are triturated and collected by centrifugation, resuspended in Dulbecco's modified Eagle's medium (DMEM)/F12 medium (Gibco BRL, Invitrogen Life Technologies, Carlsbad, Calif.) containing 25 μg/ml of insulin, 100 μg/ml of transferrin, 60 μM putrescine, 30 nM selenium, 20 nM progesterone, 100 U/ml of penicillin, 100 μg/ml of streptomycin, 0.05 M Hepes, and 10% FBS. Dissociated primary retinal cells are plated onto Poly-D-lysine- and Matrigel- (BD, Franklin Lakes, N.J.) coated glass coverslips that are placed in 24-well tissue culture plates (Falcon Tissue Culture Plates, Fisher Scientific, Pittsburgh, Pa.). Cells are maintained in culture for 5 days to one month in 0.5 ml of media (as above, except with only 1% FBS) at 37° C. and 5% CO₂.

### Immunocytochemistry Analysis

The retinal neuronal cells are cultured for 1, 3, 6, and 8 weeks, and the cells are analyzed by immunohistochemistry at each time point. Immunocytochemistry analysis is performed according to standard techniques known in the art. Rod photoreceptors are identified by labeling with a rhodopsin-specific antibody (mouse monoclonal, diluted 1:500; Chemicon, Temecula, Calif.). An antibody to mid-weight neurofilament (NFM rabbit polyclonal, diluted 1:10,000, Chemicon) is used to identify ganglion cells; an antibody to β3-tubulin (G7121 mouse monoclonal, diluted 1:1000, Promega, Madison, Wis.) is used to generally identify interneurons and ganglion cells, and antibodies to calbindin (AB1778 rabbit polyclonal, diluted 1:250, Chemicon) and calretinin (AB5054 rabbit polyclonal, diluted 1:5000, Chemicon) are used to identify subpopulations of calbindin- and calretinin-expressing interneurons in the inner nuclear layer. Briefly, the retinal cell cultures are fixed with 4% paraformaldehyde (Polysciences, Inc, Warrington, Pa.) and/or ethanol, rinsed in Dulbecco's phosphate buffered saline (DPBS), and incubated with primary antibody for 1 hour at 37° C. The cells are then rinsed with DPBS, incubated with a secondary antibody (Alexa 488- or Alexa 568-conjugated secondary antibodies (Molecular Probes, Eugene, Oreg.)), and rinsed with DPBS. Nuclei are stained with 4′,6-diamidino-2-phenylindole (DAPI, Molecular Probes), and the cultures are rinsed with DPBS before removing the glass coverslips and mounting them with Fluoromount-G (Southern Biotech, Birmingham, Ala.) on glass slides for viewing and analysis.

Survival of mature retinal neurons after varying times in culture is indicated by the histochemical analyses. Photoreceptor cells are identified using a rhodopsin antibody; ganglion cells are identified using an NFM antibody; and amacrine and horizontal cells are identified by staining with an antibody specific for calretinin.

Cultures are analyzed by counting rhodopsin-labeled photoreceptors and NFM-labeled ganglion cells using an Olympus IX81 or CZX41 microscope (Olympus, Tokyo, Japan). Twenty fields of view are counted per coverslip with a 20× objective lens. Six coverslips are analyzed by this method for each condition in each experiment. Cells that are not exposed to any stressor are counted, and cells exposed to a stressor are normalized to the number of cells in the control.

### Example 5

### Effect of Retinylamine Compounds on Retinal Cell Survival

This Example describes the use of the mature retinal cell culture system that comprises a cell stressor for determining the effects of a retinylamine derivative compound on the viability of the retinal cells.

Retinal cell cultures are prepared as described in Example 2. A2E is added as a retinal cell stressor. After culturing the cells for 1 week, a chemical stress, A2E, is applied. A2E is diluted in ethanol and added to the retinal cell cultures at concentration of 0, 10 μM, 20 μM, and 40 μM. Cultures are treated for 24 and 48 hours. A2E is obtained from Dr. Koji Nakanishi (Columbia University, New York City, N.Y.) or is synthesized according to the method of Parish et al. (Proc. Natl. Acad. Sci. USA 95:14602-13 (1998)). A retinylamine derivative compound is then added to the culture. To other retinal cell cultures, a retinylamine derivative compound is added before application of the stressor or is added at the same time that A2E is added to the retinal cell culture. The cultures are maintained in tissue culture incubators for the duration of the stress at 37° C. and 5% CO₂. The cells are then analyzed by immunocytochemistry as described in Example 1.

### Apoptosis Analysis

Retinal cell cultures are prepared as described in Example 1 and cultured for 2 weeks and then exposed to white light stress at 6000 lux for 24 hours followed by a 13-hour rest period. A device was built to uniformly deliver light of specified wavelengths to specified wells of the 24-well plates. The device contained a fluorescent cool white bulb (GE P/N FC 12T9/CW) wired to an AC power supply. The bulb is mounted inside a standard tissue culture incubator. White light stress is applied by placing plates of cells directly underneath the fluorescent bulb. The CO₂ levels are maintained at 5%, and the temperature at the cell plate is maintained at 37° C. The temperature was monitored by using thin thermocouples. The light intensities for all devices were measured and adjusted using a light meter from Extech Instruments Corporation (P/N 401025; Waltham, Mass.). A retinylamine derivative compound is added to wells of the culture plates prior to exposure of the cells to white light and is added to other wells of the cultures after exposure to white light. To assess apoptosis, TUNEL is performed as described herein.

Apoptosis analysis is also performed after exposing retinal cells to blue light. Retinal cell cultures are cultured as described in Example 1. After culturing the cells for 1 week, a blue light stress is applied. Blue light is delivered by a custom-built light-source, which consists of two arrays of 24 (4×6) blue light-emitting diodes (Sunbrite LED P/N SSP-01TWB7UWB12), designed such that each LED is registered to a single well of a 24 well disposable plate. The first array is placed on top of a 24 well plate full of cells, while the second one is placed underneath the plate of cells, allowing both arrays to provide a light stress to the plate of cells simultaneously. The entire apparatus is placed inside a standard tissue culture incubator. The CO₂ levels are maintained at 5%, and the temperature at the cell plate is maintained at 37° C. The temperature is monitored with thin thermocouples. Current to each LED is controlled individually by a separate potentiometer, allowing a uniform light output for all LEDs. Cell plates are exposed to 2000 lux of blue light stress for either 2 hours or 48 hours, followed by a 14-hour rest period. A retinylamine derivative compound is added to wells of the culture plates prior to exposure of the cells to blue light and is added to other wells of the cultures after exposure to blue light. To assess apoptosis, TUNEL is performed as described herein.

To assess apoptosis, TUNEL is performed according to standard techniques practiced in the art and according to the manufacturer's instructions. Briefly, the retinal cell cultures are first fixed with 4% paraformaldehyde and then ethanol, and then rinsed in DPBS. The fixed cells are incubated with TdT enzyme (0.2 units/μl final concentration) in reaction buffer (Fermentas, Hanover, Md.) combined with Chroma-Tide Alexa568-5-dUTP (0.1 μM final concentration) (Molecular Probes) for 1 hour at 37° C. Cultures are rinsed with DPBS and incubated with primary antibody either overnight at 4° C. or for 1 hour at 37° C. The cells are then rinsed with DPBS, incubated with Alexa 488-conjugated secondary antibodies, and rinsed with DPBS. Nuclei are stained with DAPI, and the cultures are rinsed with DPBS before removing the glass coverslips and mounting them with Fluoromount-G on glass slides for viewing and analysis.

Cultures are analyzed by counting TUNEL-labeled nuclei using an Olympus IX81 or CZX41 microscope (Olympus, Tokyo, Japan). Twenty fields of view are counted per coverslip with a 20× objective lens. Six coverslips are analyzed by this method for each condition. Cells that are not exposed to a retinylamine derivative compound are counted, and cells exposed to the antibody are normalized to the number of cells in the control. Data are analyzed using the unpaired Student's t-test.

When ranges are used herein for physical properties, such as molecular weight, or chemical properties, such as chemical formulae, all combinations and subcombinations of ranges and specific embodiments therein are intended to be included.

From the foregoing it will be appreciated that, although specific embodiments have been described herein for purposes of illustration, various modifications may be made without deviating from the spirit and scope of the invention. Those skilled in the art will recognize, or be able to ascertain, using no more than routine experimentation, many equivalents to the specific embodiments described herein. Such equivalents are intended to be encompassed by the following claims.

1. A method of treating an ophthalmic disease or disorder in a subject, wherein the ophthalmic disease or disorder is selected from diabetic retinopathy, diabetic maculopathy, diabetic macular edema, retinal ischemia, ischemia-reperfusion related retinal injury, and metabolic optic neuropathy, said method comprising administering to the subject a composition that comprises a retinylamine derivative and a pharmaceutically acceptable carrier, wherein the retinylamine derivative is a compound having the structure of formula I: 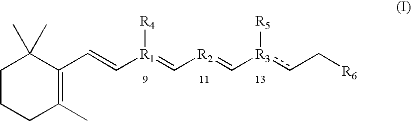
or a stereoisomer, prodrug, pharmaceutically acceptable salt, hydrate, solvate, acid salt hydrate, N-oxide or isomorphic crystalline form thereof,
wherein R₁ and R₃ are independently C;
wherein R₂ is CH;
wherein R₄ and R₅ are each independently H, saturated or unsaturated lower alkyl, C₃ to C₄ cycloalkyl —CH₂—NR₇R₈;
wherein R₆ is H, saturated or unsaturated C₁ to C₁₄ alkyl, C₃ to C₁₀ cycloalkyl, halogen, heterocycle, —CH₂—NR₇R₈, —NR₇R₈, or —NR₇R₈R₉⁺;
wherein R₇, R₈, and R₉ are each independently H, saturated or unsaturated lower alkyl, C₃ to C₄ cycloalkyl, —OH, or —OR₁₀, and wherein R₁₀ is a saturated lower alkyl.

2. (canceled)

3. (canceled)

4. (canceled)

5. (canceled)

6. (canceled)

7. The method of claim 1, wherein each of R₁ and R₃ is C, and R₂ is CH, and wherein R₆ is —NR₇R₈ or —NR₇R₈R₉⁺.

8. The method of claim 1 wherein each of R₄ and R₅ is a lower alkyl and R₆ is —NR₇R₈ or —NR₇R₈R₉⁺.

9. (canceled)

10. The method of claim 1, wherein each of R₁ and R₃ is C, and R₂ is CH and the retinylamine derivative compound has the following structure of formula I(B): 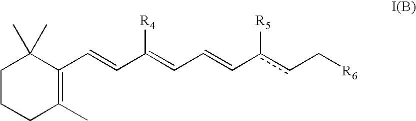
wherein R₄ and R₅ are each independently H, saturated or unsaturated lower alkyl, C₃ to C₄ cycloalkyl, —CH₂—SR₇R₈⁺, —CH₂—NR₇R₈, —NR₇R₈, or —NR₇R₈R₉⁺;
wherein R₆ is H, saturated or unsaturated C₁ to C₁₄ alkyl, C₃ to C₁₀ cycloalkyl, halogen, heterocycle, —CH₂—NR₇R₈, —NR₇R₈, or —NR₇R₈R₉⁺;
wherein R₇, R₈, and R₉ are each independently H, saturated or unsaturated lower alkyl, C₃ to C₄ cycloalkyl, —OH, or —OR₁₀, and wherein R₁₀ is a saturated lower alkyl.

11. (canceled)

12. The method of claim 10 wherein each of R₄ and R₅ is a lower alkyl and R₆ is —NR₇R₈ or —NR₇R₈R₉⁺.

13. (canceled)

14. The method of claim 1, wherein the retinylamine derivative compound inhibits an isomerization step of the retinoid cycle.

15. The method of claim 1 wherein the retinylamine derivative is selected from an all trans-isomer, a 9-cis-isomer, an 11-cis-isomer, a 13-cis-isomer, a 9,11-di-cis-isomer, a 9,13-di-cis-isomer, a 11,13-di-cis-isomer, and a 9,11,13-tri-cis-isomer.

16. The method of claim 1 wherein the retinylamine derivative is 11-cis retinylamine.

17. The method of claim 1 wherein the retinylamine derivative is selected from 9-cis retinylamine, 13-cis retinylamine, and all trans retinylamine.

18. The method of claim 1 wherein the retinylamine derivative is a compound having a structure selected from the following structures I(a)-10):

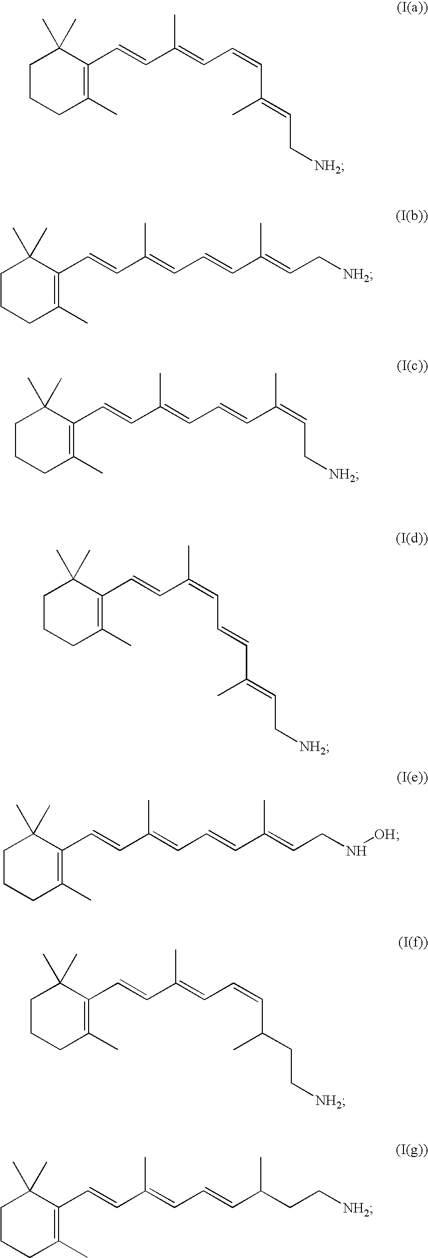

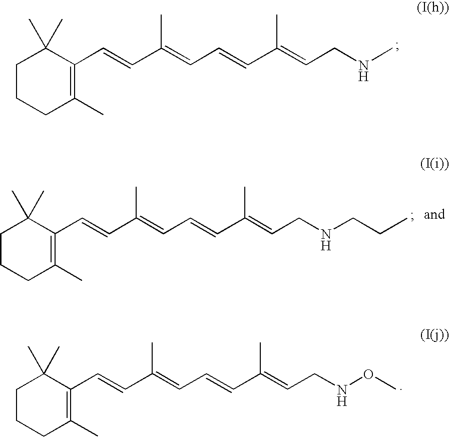

19. (canceled)

20. A method of treating an ophthalmic disease or disorder in a subject, wherein the ophthalmic disease or disorder is selected from diabetic retinopathy, diabetic maculopathy, diabetic macular edema, retinal ischemia, ischemia-reperfusion related retinal injury, and metabolic optic neuropathy, said method comprising administering to the subject a composition that comprises a retinylamine derivative and a pharmaceutically acceptable carrier, wherein the retinylamine derivative is a compound having the structure of formula II: 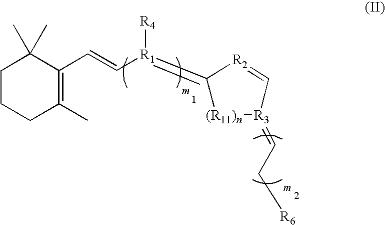
or a stereoisomer, prodrug, pharmaceutically acceptable salt, hydrate, solvate, acid salt hydrate, N-oxide or isomorphic crystalline form thereof,
wherein n is 1, 2, 3, or 4;
m₁ plus m₂ equals 1, 2, or 3; and
wherein R₁ and R₃ are each C; R₂ is CH, or N; and R₁₁ is C(H₂);
wherein R₄ is H, saturated or unsaturated lower alkyl, C₃ to C₄ cycloalkyl, —CH₂—NR₇R₈;
wherein R₆ is H, guanidinium, isouronium, —CH₂—NR₇R₈, —NR₇R₈, or NR₇R₈R₉⁺;
wherein R₇, R₈, and R₉ are each independently H, saturated or unsaturated lower alkyl, C₃ to C₄ cycloalkyl, —OH, or —OR₁₀, and wherein R₁₀ is a saturated lower alkyl.

21. (canceled)

22. (canceled)

23. (canceled)

24. (canceled)

25. (canceled)

26. (canceled)

27. (canceled)

28. (canceled)

29. The method according to claim 20 wherein the retinylamine derivative is a compound having the structure of formula III: 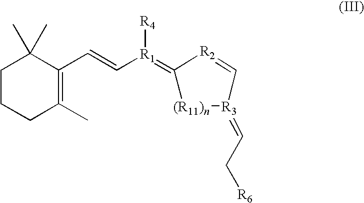
or a stereoisomer, prodrug, pharmaceutically acceptable salt, hydrate, solvate, acid salt hydrate, N-oxide or isomorphic crystalline form thereof,
wherein n is 1, 2, 3, or 4; and
wherein R₁ and R₃ are each C; R₂ is CH, or N; and R₁₁ is C(H₂);
wherein R₄ is H, saturated or unsaturated lower alkyl, C₃ to C₄ cycloalkyl, —CH₂—NR₇R₈;
wherein R₆ is H, saturated or unsaturated C₁ to C₁₄ alkyl, C₃ to C₁₀ cycloalkyl, halogen, heterocycle, guanidinium, isouronium, —CH₂—NR₇R₈, —NR₇R₈, or —NR₇R₈R₉⁺,
and wherein R₇, R₈, and R₉ are each independently H, saturated or unsaturated lower alkyl, C₃ to C₄ cycloalkyl, —OH, or —OR₁₀, and wherein R₁₀ is a saturated lower alkyl.

30. (canceled)

31. (canceled)

32. (canceled)

33. (canceled)

34. (canceled)

35. (canceled)

36. The method of claim 29 wherein each of R₁ and R₃ is C, R₂ is CH, and R₁₁ is C(H₂), and wherein R₆ is —NR₇R₈ or —NR₇R₈R₉⁺.

37. The method of claim 29 wherein each of R₁ and R₃ is C, R₂ is CH, and R₁₁ is C(H₂);
wherein R₄ is H, lower alkyl, C₃ to C₄ cycloalkyl;
wherein R₆ is H, saturated or unsaturated C₁ to C₁₄ alkyl, C₃ to C₁₀ cycloalkyl, halogen, heterocycle —CH₂—NR₇R₈, —NR₇R₈, or —NR₇R₈R₉⁺;
and wherein R₇, R₈, and R₉ are independently H, saturated or unsaturated lower alkyl, C₃ to C₄ cycloalkyl, —OH, or —OR₁₀, and wherein R₁₀ is a saturated lower alkyl.

38. (canceled)

39. The method of claim 29 wherein the retinylamine derivative is 11-cis locked retinylamine.

40. (canceled)

41. A method of treating an ophthalmic disease or disorder in a subject, wherein the ophthalmic disease or disorder is selected from diabetic retinopathy, diabetic maculopathy, diabetic macular edema, retinal ischemia, ischemia-reperfusion related retinal injury, and metabolic optic neuropathy, said method comprising administering to the subject a composition that comprises a retinylamine derivative and a pharmaceutically acceptable carrier, wherein the retinylamine derivative is a compound of formula IV: 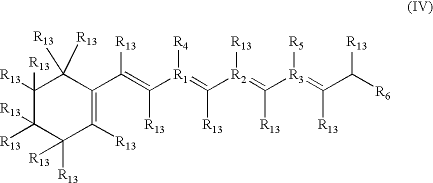
or a stereoisomer, prodrug, pharmaceutically acceptable salt, hydrate, solvate, acid salt hydrate, N-oxide or isomorphic crystalline form thereof,
wherein each R₁₃ is independently hydrogen, saturated or unsaturated C₁ to C₁₄ alkyl, C₃ to C₁₀ cycloalkyl, halogen, heterocycle, —OR₁₄, —SR₁₄, or —NR₁₄R₁₅, and wherein R₁₄ and R₁₅ are each independently H or saturated lower alkyl;
wherein R₁, R₂, and R₃ are each C;
wherein R₄ and R₅ are each independently H, saturated or unsaturated lower alkyl, C₃ to C₄ cycloalkyl, disubstituted imidazolium, trisubstituted imidazolium, pyridinium, pyrrolidinium, —CH₂—NR₇R₈;
wherein R₆ is H, saturated or unsaturated C₁ to C₁₄ alkyl, C₃ to C₁₀ cycloalkyl, halogen, heterocycle, guanidinium, isouronium, —CH₂—NR₇R₈, —NR₇R₈, or —NR₇R₈R₉⁺;
wherein R₇, R₈, and R₉ are each independently H, saturated or unsaturated lower alkyl, C₃ to C₄ cycloalkyl, —OH, or —OR₁₀, and wherein R₁₀ is saturated lower alkyl.

42. (canceled)

43. (canceled)

44. (canceled)

45. The method of claim 41 wherein each of R₁, R₂, and R₃ is C; and wherein R₆ is —NR₇R₈ or —NR₇R₈R₉⁺.

46. The method of claim 41 wherein each R₁₃ is independently hydrogen, saturated or unsaturated C₁ to C₁₄ alkyl, C₃ to C₁₀ cycloalkyl, halogen, heterocycle, —OR₁₄, —SR₁₄, or —NR₁₄R₁₅, and wherein R₁₄ and R₁₅ are each independently H or saturated lower alkyl;
wherein R₁, R₂, and R₃ are C;
wherein R₄ and R₅ are each independently H, C₁ to C₆ alkyl, C₃ to C₄ cycloalkyl, —CH₂—NR₇R₈;
wherein R₆ is H, saturated or unsaturated C₁ to C₁₄ alkyl, C₃ to C₁₀ cycloalkyl, halogen, heterocycle, —CH₂—NR₇R₈, —NR₇R₈, or —NR₇R₈R₉⁺;
wherein R₇, R₈, and R₉ are each independently H, saturated or unsaturated lower alkyl, C₃ to C₄ cycloalkyl, —OH, or —OR₁₀, and wherein R₁₀ is saturated lower alkyl;
with the proviso that at least one of R₄, R₅, and R₆ is —NR₇R₈ or —NR₇R₈R₉⁺.

47. (canceled)

48. The method of claim 41, wherein the retinylamine derivative is selected from an all trans-isomer, a 9-cis-isomer; a 11-cis-isomer; a 13-cis-isomer; a 9,11-di-cis-isomer; a 9,13-di-cis-isomer; a 11,13-di-cis-isomer; and a 9,11,13-tri-cis-isomer.

49. (canceled)

50. A method of treating an ophthalmic disease or disorder in a subject, wherein the ophthalmic disease or disorder is selected from diabetic retinopathy, diabetic maculopathy, diabetic macular edema, retinal ischemia, ischemia-reperfusion related retinal injury, and metabolic optic neuropathy, said method comprising administering to the subject a composition that comprises a retinylamine derivative and a pharmaceutically acceptable carrier, wherein the retinylamine derivative is a compound of formula V: 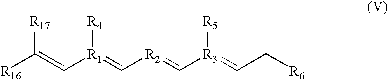
or a stereoisomer, prodrug, pharmaceutically acceptable salt, hydrate, solvate, acid salt hydrate, N-oxide or isomorphic crystalline form thereof,
wherein each of R₁₆ and R₁₇ is independently substituted or unsubstituted lower alkyl, hydroxyl, or alkoxy;
wherein R₁ and R₃ are each C;
wherein R₂ is CH;
wherein R₄ and R₅ are each independently H, saturated or unsaturated lower alkyl, C₃ to C₄ cycloalkyl, —CH₂—NR₇R₈;
wherein R₆ is H, C₁ to C₁₄ alkyl, C₃ to C₁₀ cycloalkyl, halogen, heterocycle, guanidinium, isouronium, —CH₂—NR₇R₈, —NR₇R₈, or —NR₇R₈R₉⁺;
wherein R₇, R₈, and R₉ are independently H, saturated or unsaturated lower alkyl, C₃ to C₄ cycloalkyl, —OH, or —OR₁₀, and wherein R₁₀ is a saturated lower alkyl.

51. (canceled)

52. (canceled)

53. (canceled)

54. (canceled)

55. (canceled)

56. The method of claim 50 wherein each of R₁ and R₃ is C and R₂ is CH; and wherein R₆ is —NR₇R₈ or —NR₇R₈R₉⁺.

57. The method according to claim 50 wherein each of R₁₆ and R₁₇ is independently substituted or unsubstituted lower alkyl, hydroxyl, alkoxy;
wherein each of R₁ and R₃ is C and R₂ is CH;
wherein R₄ and R₅ are each independently H, saturated or unsaturated lower alkyl, C₃ to C₄ cycloalkyl, —CH₂—NR₇R₈;
wherein R₆ is H, saturated or unsaturated C₁ to C₁₄ alkyl, C₃ to C₁₀ cycloalkyl, halogen, heterocycle, —CH₂—NR₇R₈, —NR₇R₈, or —NR₇R₈R₉⁺;
wherein R₇, R₈, and R₉ are independently H, saturated or unsaturated lower alkyl, C₃ to C₄ cycloalkyl, —OH, or —OR₁₀, and wherein R₁₀ is saturated lower alkyl.

58. (canceled)

59. The method of claim 50 wherein the retinylamine derivative is the compound 10-ethyl-3,7-dimethyl-dodeca-2,4,6,8-tetraenylamine having the structure (V(a)):

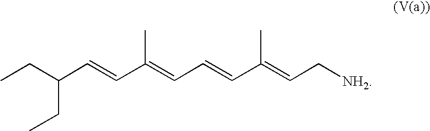

60. (canceled)

61. The method according to any one of claims 1, 20, 41, and 50 wherein accumulation of lipofuscin pigment is inhibited in an eye of the subject.

62. (canceled)

63. The method according to any one of claims 1, 20, 41, and 50 wherein the retinylamine derivative is administered by a route selected from by mouth, by eye drops, by intraocular injection, or by periocular injection.

64. (canceled)

65. (canceled)

66. The method according to any one of claims 1, 20, 41, and 50 wherein degeneration of a retinal cell is inhibited, wherein said retinal cell is selected from a retinal neuronal cell, a photoreceptor cell, an amacrine cell, a horizontal cell, a ganglion cell, and a bipolar cell.

67. (canceled)

68. (canceled)
